(19) United States
(12) Patent Application Publication
(10) Pub. No.: US 2020/0262799 A1
(43) Pub. Date: Aug. 20, 2020

(54) PROCESS FOR PREPARING FUNCTIONALIZED 1,2,4,5-TETRAZINE COMPOUNDS

(71) Applicant: UNIVERSITE DE BOURGOGNE, Dijon (FR)

(72) Inventors: Jean-Cyrille HIERSO, Dijon (FR); Julien ROGER, Dijon (FR); Richard DECREAU, Dijon (FR); Christelle TESTA, Villeurbanne (FR)

(73) Assignee: Universite de Bourgogne, Dijon (FR)

(21) Appl. No.: 15/774,564

(22) PCT Filed: Nov. 29, 2016
(86) PCT No.: PCT/EP2016/079154

§ 371 Date: May 8, 2018

Related U.S. Application Data
(60) Provisional application No. 62/260,741, filed on Nov. 30, 2015.

(51) Int. Cl. C07D 257/08; B01J 23/44

(52) U.S. Cl. CPC C07D 257/08; B01J 23/44

(57) ABSTRACT

The present invention relates to a process for the synthesis of 3,6 functionalized 1,2,4,5-tetrazine compounds.

The present invention relates to a process for the synthesis of 3,6 functionalized 1,2,4,5-tetrazine compounds of general formula (I).

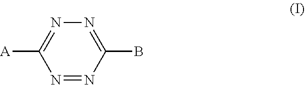

as defined thereafter.

The chemistry of s-tetrazines (1,2,4,5-tetrazines) has attracted increasing interest over the years, owing to multiple applications (biochemical, materials) in relation with their unique physicochemical properties. Significant improvements in the tetrazine synthesis have been reported. However, practical synthetic methods for the functionalization of tetrazine, such as transition metal catalyzed methods, are scarce.

The palladium-catalyzed cross-coupling reactions of aromatics for C—C bond formation were recently adapted to the tetrazine series in a very limited scope. The tetrazine ring may act as a ligand for metals, hence poisoning the catalytic activity. Moreover, tetrazines may be reduced by metals and subsequent ring opening may occur.

However, recent works from the Devaraj group described a practical palladium-catalyzed Heck-type reaction for producing alkenyl tetrazines: the combination of hanging mesityl functions and a careful optimization of the conditions allow to operate the palladium chemistry with excellent tolerance.

Therefore, there remains a need for the development of new methods of synthesis of functionalized 3,6-diphenyl-1,2,4,5-tetrazine. Such methods should be an efficient and practical entry to further access highly substituted 3,6-diphenyl-1,2,4,5-tetrazine derivatives in order to facilitate the development and applications of conjugated tetrazines, including their late stage short time fluorination.

Ligand directed C—H bond activation/functionalization by a transition metal has emerged as a powerful method for selectively creating C—C and C—X bonds (X═N, O, S, halogen). However, C—H activation reaction appeared as one of the most challenging model reaction for substituted tetrazines such as 3,6-diphenyl-1,2,4,5-tetrazine 1: they could be reduced by metals then undergo decomposition, and the selectivity may be affected by the presence of up to four sp2C— H bonds in ortho-position of the heteroaromatic ring.

As a result of intensive research conducted for the development of new methods of synthesis of functionalized 3,6-diphenyl-1,2,4,5-tetrazine, the Applicant found that functionalized 3,6-diphenyl-1,2,4,5-tetrazine may be obtained by catalyzed direct C—H functionalization of 3,6-diphenyl-1,2,4,5-tetrazines and with the introduction of various useful functional groups, such as halides. Introducing halogen atoms on the aryl ring is a first step towards further extension of the conjugation length and the building of more sophisticated structures through the use of metal-catalyzed coupling reactions.

The process is carried out by direct functionalization of one or more C—H bonds and thus allows the introduction of reactive functional groups such as bromo, chloro, iodo, fluoro and acetate without prefunctionalization (metalation or mesitylation). From these halogenated compounds, large numbers of various molecules can be built and direct fluorination of heterocyclic compounds is highly recoverable.

This invention relates to a process for producing a compound of formula (I),

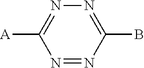

wherein
A is

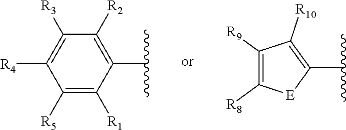

B is

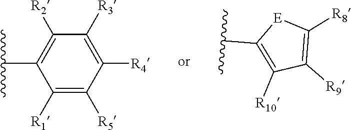

A and B being the same;
R₁, R₁′, R₂ and R₂′ may be the same or different and represent each a hydrogen atom, a halogen atom, a substituted or unsubstituted group selected from alkyl, cycloalkyl, aryl, alkyloxy, alkyloxycarbonyl, cycloalkyloxy, aryloxy, alkylamino, cycloalkylamino, arylamino, provided that at least one of R₁, R₁′, R₂ and R₂′ is a halogen atom or acetate group;
R₃, R₃′, R₄, R₄′, R₅, R₅′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, arylamino;
R₈, R₈′, R₉, R₉′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, and arylamino;
R₁₀, R₁₀′ may be the same or different and represent each a hydrogen atom or a halogen atom, provided that at least one of R₁₀ and R₁₀′ is a halogen atom or acetate group;
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;

said process comprising:

reacting a compound of formula (II)

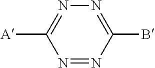

wherein
A′ is

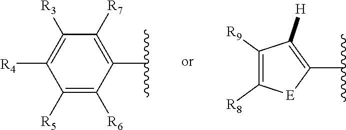

B′ is

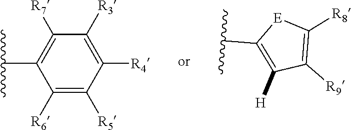

A′ and B′ being the same;
R₆, R₆′, R₇ and R₇′ may be the same or different and represent each a hydrogen atom, a halogen atom, a substituted or unsubstituted group selected from alkyl, cycloalkyl, aryl, alkyloxy, cycloalkyloxy, aryloxy, alkylamino, cycloalkylamino, and arylamino, provided that at least one of R₆, R₆′, R₇ and R₇′ is a hydrogen atom;
R₃, R₃′, R₄, R₄′, R₅, R₅′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino, and arylamino;
R₈, R₈′, R₉, R₉′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino and arylamino;
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
   with an oxidative reagent in presence of a catalyst.

In the present invention, when A and B are respectively

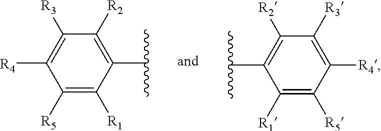

then the compounds are named (Ia) while when A and B are respectively

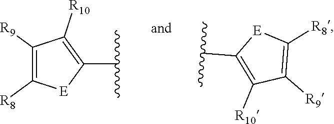

then the compounds are named (Ib).

As used herein, halogen means an atom selected from bromine, chlorine, fluorine and iodine.

“Alkyl group” means a straight chain or branched hydrocarbon chain having 1 to 10 carbon atoms, preferably 1 to 6, more preferably 2 to 5 and optionally having at least one double bonds. Exemplary alkyl groups include but are not limited to methyl, ethyl, propyl, butyl, isopropyl, isobutyl, isopentyl, neopentyl, tert-butyl, n-hexyl, heptyl, octyl, nonyl, decyl, ethenyl, and propenyl.

“Cycloalkyl group” means an cyclic alkyl group with 3 to 20 carbon atoms, preferably 3 to 10, more preferably 3 to 6, having a single cyclic ring or multiple condensed rings optionally said ring or said rings having at least one double bonds. Exemplary cycloalkyl groups include but are not limited to cyclopropyl, cyclobutyl, cyclopentyl, cyclohexyl, adamantyl and cyclopentenyl.

“Alkyloxy group or alkoxy group” is a moiety of formula —ORₓ with Rₓ is an “alkyl group” as defined above. Exemplary alkyloxy groups include but are not limited to methoxy, ethoxy, propyloxy, butyloxy, hexyloxy, isopropyloxy, isobutyloxy, neopentyloxy, tert-butyloxy.

“Cycloalkyloxy group or cycloalkoxy group” is a moiety of formula —OR_{y} with R_{y} is a “cycloalkyl group” as defined above. Exemplary cycloalkyloxy groups include but are not limited to cyclopropyloxy, cyclobutyloxy, cyclopentyloxy, cyclohexyloxy and the like.

“Alkylamino group” means a —NRₐR_{b} wherein Rₐ and R_{b} are each independently of the other an alkyl group as defined above. Exemplary alkylamino groups include but are not limited to —N(CH₃)₂, —N(CH₃)(CH₂CH₃) and the like.

“Cycloalkylamino group” means a —NR_{c}R_{d} wherein R_{c} and R_{d} constitute a cycloalkyl group as defined above.

“Aryl group” means any functional group or substituent derived from an aromatic or an heteroaromatic ring including O and S heteroatoms, such as not exhaustively and for example: phenyl, biphenyl, naphthyl, furyl, thienyl, benzofuryl, benzothienyl, etc.

“Alkyloxycarbonyl group”, is a moiety of formula —COORₓ with Rₓ is an “alkyl group” as defined above. Exemplary alkyloxycarbonyl groups include but are not limited to acetate, ethyloxycarbonyle and the like.

“Aryloxy group” is a moiety of formula —ORₑ wherein Rₑ is an aryl group as defined above. “Arylamino radical” means a —NR_{f}R_{g} wherein R_{f} and R_{g} are each independently of the other an aryl group as defined above.

According to one embodiment the process leads to a compound of formula (Ia),

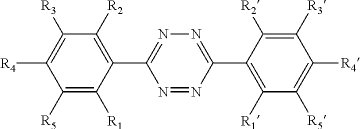

wherein
R₁, R₁′, R₂ and R₂′ may be the same or different and represent each a hydrogen atom, a halogen atom, a substituted or unsubstituted group selected from alkyl, cycloalkyl, aryl, alkyloxy, cycloalkyloxy, aryloxy, alkylamino, cycloalkylamino, arylamino, provided that at least one of R₁, R₁′, R₂ and R₂′ is a halogen atom or acetate group;
R₃, R₃′, R₄, R₄′, R₅, R₅′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, arylamino;
   said process comprising:
   reacting a compound of formula (IIa)

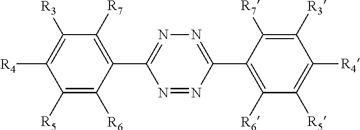

wherein
R₆, R₆′, R₇ and R₇′ may be the same or different and represent each a hydrogen atom, a halogen atom, a substituted or unsubstituted group selected from alkyl, cycloalkyl, aryl, alkyloxy, cycloalkyloxy, aryloxy, alkylamino, cycloalkylamino, and arylamino, provided that at least one of R₆, R₆′, R₇ and R₇′ is a hydrogen atom;
R₃, R₃′, R₄, R₄′, R₅, R₅′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino, and arylamino;
   with an oxidative reagent in presence of a catalyst.

According to one embodiment, R₁, R₁′, R₂ and R₂′ may be the same or different and represent each a hydrogen atom, an acetate group or a halogen atom, provided that at least one of R₁, R₁′, R₂ and R₂′ is a halogen atom or an acetate group. According to one embodiment, R₁, R₁′, R₂ and R₂′ may be the same or different and represent each a hydrogen atom, an acetate group or a halogen atom, provided that at least two of R₁, R₁′, R₂ and R₂′ is a halogen atom or an acetate group. According to one embodiment, R₁, R₁′, R₂ and R₂′ may be the same or different and represent each a hydrogen atom, an acetate group or a halogen atom, provided that at least three of R₁, R₁′, R₂ and R₂′ is a halogen atom or an acetate group. According to one embodiment, R₁, R₁′, R₂ and R₂′ represent each a halogen atom or an acetate group. According to another embodiment, R₁, R₁′, R₂ and R₂′ may be the same or different and represent each a hydrogen atom or a halogen atom, provided that at least one of R₁, R₁′, R₂ and R₂′ is a halogen atom. According to another embodiment, R₁, R₁′, R₂ and R₂′ may be the same or different and represent each a hydrogen atom or a halogen atom, provided that at least two of R₁, R₁′, R₂ and R₂′ is a halogen atom. According to another embodiment, R₁, R₁′, R₂ and R₂′ may be the same or different and represent each a hydrogen atom or a halogen atom, provided that at least three of R₁, R₁′, R₂ and R₂′ is a halogen atom. According to another embodiment, R₁, R₁′, R₂ and R₂′ may be the same or different and represent each a halogen atom.

According to one embodiment of the present invention, when R₁, R₁′, R₂ and R₂′ are each a halogen atom, the halogen atom being the same or different, and is selected from fluorine, chlorine, bromine and iodine, provided that at least one of them is a different halogen atom compared to the others. In this embodiment, the compounds are highly unequally polyfunctionalized compounds of formula (I) and it will be referred herein after as “unequally polyhalogenated”. For example one is fluorine and the others are bromine, or two are chlorine and two are iodine.

According to one embodiment, R₃, R₃′, R₄, R₄′, R₅ and R₅′ represent each a hydrogen atom.

According to one embodiment, R₆, R₆′, R₇ and R₇′ may be the same or different and represent each a hydrogen atom or a halogen atom, provided that at least one of R₆, R₆′, R₇ and R₇′ is a hydrogen atom. According to one embodiment, R₆, R₆′, R₇ and R₇′ may be the same or different and represent each a hydrogen atom or a halogen atom, provided that at least two of R₆, R₆′, R₇ and R₇′ is a hydrogen atom. According to one embodiment, R₆, R₆′, R₇ and R₇′ may be the same or different and represent each a hydrogen atom or a halogen atom, provided that at least three of R₆, R₆′, R₇ and R₇′ is a hydrogen atom.

According to a second embodiment the process leads to a compound of formula (Ib),

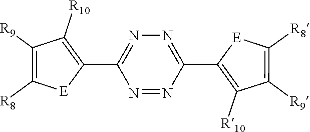

wherein
R₈, R₈′, R₉, R₉′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, and arylamino;
R₁₀, R₁₀′ may be the same or different and represent each a hydrogen atom or a halogen atom, provided that at least one of R₁₀ and R₁₀′ is a halogen atom or acetate group;
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
   said process comprising:
   reacting a compound of formula (IIb)

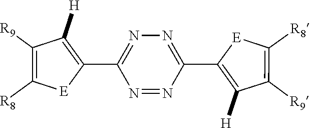

wherein
R₈, R₈′, R₉, R₉′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino and arylamino;
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
   with an oxidative reagent in presence of a catalyst.

According to one embodiment, R₁₀ and R₁₀′ represent each an acetate group or a halogen atom. According to one embodiment, R₁₀ and R₁₀′ represent each an acetate group, chlorine, bromine, fluorine or iodine.

According to one embodiment, R₈, R₈′, R₉, R₉′ represent each a hydrogen atom.

According to the present invention, the definitions given hereunder apply to all compounds (Ia), (Ib), (IIa) and (IIb).

According to one embodiment, the halogen atom is selected from chlorine, bromine, fluorine or iodine.

According to another embodiment, the oxidative reagent is selected from the group comprising N-chlorosuccinimide, N-bromosuccinimide, N-iodosuccinimide, N-fluorobenzenesulfonimide and (diacetoxyiodo)benzene.

According to another embodiment, the amount of oxidative reagent is ranging from 1 equivalent to 12 equivalent of compound (II). According to another advantageous embodiment, the amount of oxidant for producing a mono functionalized compound (I) is ranging from 1 equivalent to 1.1 equivalents of compound (II). According to another advantageous embodiment, the amount of oxidant for producing a tetra functionalized compound (I) is ranging from 4 equivalents to 10 equivalents of compound (II), more preferably from 5 equivalents to 7 equivalents. According to another advantageous embodiment, using microwave irradiation, the amount of oxidant for producing a di functionalized compound (I) is ranging from 3 equivalents to 4 equivalents.

According to another advantageous embodiment, the amount of oxidant for producing a tri functionalized compound (I) is ranging from 5 equivalents to 8 equivalents.

According to another advantageous embodiment, when the oxidative reagent is N-bromosuccinimide then the amount of oxidative reagent is ranging from 1 equivalent to 8 equivalent of compound (II). According to one embodiment, the amount of oxidant for producing a mono functionalized compound (I) is ranging from 1 equivalent to 1.1 equivalents of compound (II). According to one embodiment, the amount of oxidant for producing a tetra functionalized compound (I) is ranging from 4 equivalents to 8 equivalents of compound (II), preferably from 5 equivalents to 7 equivalents. According to another advantageous embodiment, using microwave irradiation, the amount of oxidant for producing a di functionalized compound (I) is ranging from 3 equivalents to 4 equivalents. According to another advantageous embodiment, the amount of oxidant for producing a tri functionalized compound (I) is ranging from 5 equivalents to 8 equivalents.

According to one embodiment, when the oxidative reagent is N-chlorosuccinimide or N-iodosuccinimide then the amount of oxidative reagent is ranging from 1 equivalent to 12 equivalent of compound (II. According to one embodiment, the amount of oxidant for producing a mono functionalized compound (I) is ranging from 1 equivalent to 1.1 equivalents of compound (II). According to one embodiment, the amount of oxidant for producing a tetra functionalized compound (I) is ranging from 8 equivalents to 12 equivalents of compound (II), preferably from 10 equivalents to 12 equivalents. According to another advantageous embodiment, using microwave irradiation, the amount of oxidant for producing a di functionalized compound (I) is ranging from 3 equivalents to 4 equivalents. According to another advantageous embodiment, the amount of oxidant for producing a tri functionalized compound (I) is ranging from 5 equivalents to 8 equivalents.

According to another advantageous embodiment, when the oxidative reagent is (diacetoxyiodo)benzene then the amount of oxidative reagent is ranging from 1 equivalent to 10 equivalent of compound (II). According to one embodiment, the amount of oxidant for producing a mono functionalized compound (I) is ranging from 1 equivalent to 1.1 equivalents of compound (II). According to another advantageous embodiment, using microwave irradiation, the amount of oxidant for producing a di functionalized compound (I) is ranging from 3 equivalents to 4 equivalents. According to another advantageous embodiment, the amount of oxidant for producing a tri functionalized compound (I) is ranging from 8 equivalents to 10 equivalents.

According to one embodiment, the catalyst is a palladium catalyst. According to a preferred embodiment, the catalyst is selected from the group comprising palladium(II) catalyst and palladium(0) catalyst. Preferably, the catalyst is selected from the group comprising palladium acetate, allylpalladium(II) chloride dimer, palladium chloride, tris(dibenzylideneacetone)dipalladium, and bis(dibenzylideneacetone)palladium. More preferably, the catalyst is selected from the group comprising palladium acetate, bis(dibenzylideneacetone)palladium and palladium chloride.

According to one embodiment, the amount of catalyst is ranging from 0.1% to 50%, more preferably 0.1% to 30%, more preferably 0.1% to 20% more preferably 1% to 50%, more preferably 5% to 20%, more preferably 8% to 15%, more preferably 1% to 15%, in mole of compound (II).

According to one embodiment, the process is carried out in presence of a polar solvent. According to a preferred embodiment, the solvent used is selected from the group comprising dichloroethane, nitromethane, trifluoromethylbenzene, acetic acid, pivalic acid and propionic acid. According to a preferred embodiment, the solvent used is dichloroethane. According to another preferred embodiment, the solvent used is acetic acid. According to another preferred embodiment, the solvent used is trifluoromethylbenzene.

According to one embodiment, the synthesis of compound (I) is performed at a temperature ranging from 80° C. to 150° C., preferably from 90° C. to 130° C., more preferably from 100° C. to 120° C.

According to one embodiment, the synthesis of compound (I) is performed under microwave irradiation.

According to one embodiment, the synthesis of compound (I) is performed for a time ranging from 1 to 20 hours, preferably from 1 to 18 hours. According to one embodiment, the synthesis of compound (I) is performed for 17 hours.

According to another embodiment, the synthesis of compound (I) is performed for a time ranging from 1 to 60 minutes, preferably from 5 to 40 minutes, more preferably for 10 minutes.

According to one embodiment, the synthesis of compound (I) is performed for 17 hours in absence of microwave irradiation. According to another embodiment, the synthesis of compound (I) under microwave irradiation is performed for a time ranging from 1 to 60 minutes, preferably from 5 to 40 minutes, more preferably for 10 minutes.

Compounds of formula (Ia) wherein R₁, R₁′, R₂ and R₂′ may be the same or different and represent each a halogen atom, the halogen atom being the same or different, provided that at least one of R₁, R₁′, R₂ and R₂′ is a different halogen atom compared to the others;

R₃, R₃′, R₄, R₄′, R₅, R₅′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, arylamino
are new and are also part of the invention.

Thus compounds 12a-12c, 13a-13b, 14a-14b, 15-43, 44a-44e, 45a-b disclosed in the examples are part of the invention.

According to one embodiment, compound (I) can be further functionalized on the tetrazine function and can be then optionally grafted on a biomolecule.

Compounds of formulae (I), either (Ia) or (Ib), prepared from radiolabeled oxidative reagents can be used in nuclear medicine and imaging.

The present invention is further illustrated by the following examples.

### EXAMPLE 1: SYNTHESIS OF COMPOUNDS OF FORMULA (I)

### Material

3,6-bis(2-fluorophenyl)-1,2,4,5-tetrazine is synthesized under the conditions developed by Clavier et al (G. Clavier, P. Audebert, Chem. Rev. 2010, 110, 3299-3314). All others reagents were purchased from commercial suppliers and used without purifications. All reactions were performed in Schlenk tubes or in a microwave reaction vessel under argon. Microwave heating was carried out using a CEM Discover microwave reactor. The microwave reactions were run in closed reaction vessels with magnetic stirring and with the temperature controlled via IR detection. ¹H (300 MHz), ¹³C (75 or 125 MHz), ¹⁹F (282 MHz) spectra were recorded on Brucker AVANCE III instrument in CDCl₃ solutions. Chemical shifts are reported in ppm relative to CDCl₃ (¹H: 7.26 and ¹³C: 77.16) and coupling constants J are given in Hz. High resolution mass spectra (HRMS) were obtained on a Thermo LTQ-Orbitrap XL with ESI source. Flash chromatography was performed on silica gel (230-400 mesh). Elemental analysis experiments were performed Thermo Electron Flash EA 1112 Series. Absorption spectra (in solution or liposome suspension) were measured on a Shimadzu UV-2550 spectrophotometer. Spectra were recorded in DCM in glass cuvettes 1×1×3 cm (1 cm path).

### Results:

### A. Optimisation of the Synthesis of Compound of Formula (Ia)

### A.1 Optimisation of C—H Monofunctionalization of 3,6-diphenyl-1,2,4,5-tetrazine (1) Under Conditions [a] or [b]

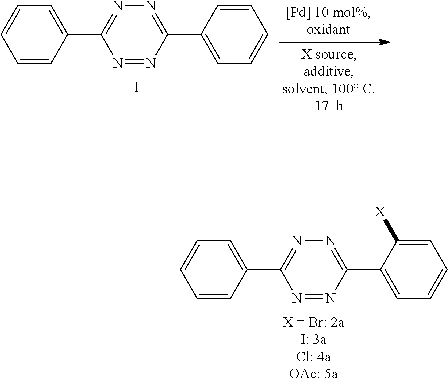

| | | Oxidant | | Conv. | 2a-5a |
|---|---|---|---|---|---|
| Entry | [Pd] | (equiv) | Solvent | (%) | (%) |
| 1 | — | NBS (1.0) | DCE | 0 | 0 |
| 2 | Pd(OAc)₂ | NBS (1.0) | DCE | 75 | 55 (48) |
| 3 | PdCl₂ | NBS (1.0) | DCE | 26 | 26 |
| 4 | Pd(dba)₂ | NBS (1.0) | DCE | 67 | 54 (45) |
| 5 | Pd(dba)₂ | NBS (1.7) | DCE | 87 | 57 |
| 6 | Pd(dba)₂ | NBS (2.2) | DCE | 96 | 41 |
| 7 | Pd(dba)₂ | NIS (1.0) | DCE | 55 | 49(33) |
| 8[b] | PdCl₂ | NCS (1.0) | HOAc | 48 | 44(32) |
| 9 | Pd(OAc)₂ | PhI(OAc)₂ | HOAc | 84 | 64(51) |
| | | (1.0) | | | |

[a] Conditions: 3,6-diphenyl-1,2,4,5-tetrazine (1) (1 equiv), [Pd] (10 mol %), X source (1.0 to 2.2 equiv), solvent (0.125M), 100° C., under argon, 17 h. ¹H NMR yield and isolated yield under bracket. DCE: dichloroethane, HOAc: Acetic acid.
[b]same as [a] except 120° C.

In the absence of palladium no reaction occurred (entry 1). The reaction of equimolar amounts of tetrazine (1) and NBS in the presence of 10 mol % of [Pd(OAc)₂] in dichloroethane (DCE) at 100° C. for 17 h converted (1) in 75%, and afforded the expected monobrominated product (2a) with two dibrominated side-products (2b) and (2c), in a [73:17:10] ratio, respectively (entry 2). Compound (2a) can be easily purified and isolated in about 50% yield. Other palladium catalysts, such as [PdCl₂] and [Pd(dba)₂] provided to lower conversions (entries 3, 4). Increasing amounts of NBS allowed greater conversions but was detrimental to the selectivity in (2a) (entries 5, 6).

Iodination of (1) was achieved using N-iodosuccinimide in the presence of 10% [Pd(dba)₂] in DCE, to afford 55% conversion yield and a [89:11] ratio of the monoiodinated product (3a) and the symmetrical diiodinated analogue (3b) (entry 7). Chlorination of (1) was achieved using N-chlorosuccinimide and 10% [PdCl₂] in HOAc at 120° C. (entry 8, conversion 48%) to afford the monochlorinated tetrazine (4a) with 92% selectivity. The scope of such an unprecedented C—H functionalization of s-tetrazine was extended to acetoxylation reactions using PhI(OAc)₂ and 10 mol % [Pd(OAc)₂] in HOAc to afford pure (5a) in 51% yield ((1) converted in 84%, entry 9).

### A.2 Optimisation of Tetra-Functionalization of 3,6-diphenyl-1,2,4,5-tetrazine (1) Under Conditions [a]

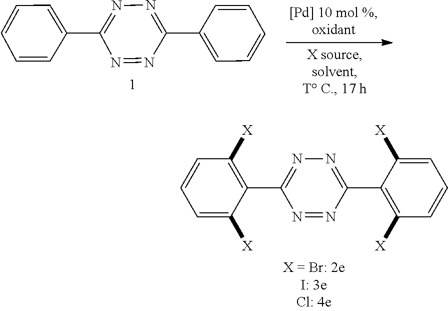

| | | Oxidant | | | |
|---|---|---|---|---|---|
| Entry | [Pd] | (equiv) | Solvent | T° C. | 2e-4e |
| 1 | Pd(dba)₂ | NBS (8.0) | HOAc | 100 | 2e: 99 (98) |
| 2 | Pd(OAc)₂ | NBS (6.0) | HOAc | 120 | 2e: 99 (89) |
| 3 | Pd(OAc)₂ | NIS (12.0) | HOAc | 120 | 3e: 71 (64) |
| 4 | Pd(OAc)₂ | NCS (10.0) | HOAc | 120 | 4e: 80 (34) |

[a] Conditions: 3,6-diphenyl-1,2,4,5-tetrazine (1) (1 equiv), [Pd] (10 mol %), NXS (6 to 12 equiv), solvent (0.125M), under argon, 17 h. ¹H NMR yield and isolated yield under bracket.

Tetrahalogenation of 3,6-diphenyl-1,2,4,5-tetrazine was achieved upon adjusting the amounts of halogenation reagent. 8 equiv of NBS and 10 mol % of Pd catalyst were necessary to achieve full conversion of (1) affording tetrabrominated tetrazine (2e) in 98% isolated yield (entry 1). Di- and trihalogenated species (2b), (2d) were also isolated when lower amounts of NBS were used. The reaction was even faster using lower amounts of NBS (6 equiv, entry 2) in the presence of 10 mol % of [Pd(OAc)₂] in HOAc at 120° C.

Using the same catalytic system, further multiple C—H halogenation reactions were successfully achieved with other N-halosuccinimides (10 equiv) albeit in lower yields (entries 3, 4): tetraiodotetrazine (3e) and tetrachlorotetrazine (4e) were obtained from NIS in 71% yield and from NCS in 80% yield, respectively.

### A.3 Optimisation of Fluorination of 3,6-diphenyl-1,2,4,5-tetrazine (1) Toward Fast-Time Good Selectivity in Monofluorinated Product (6a) Under Conditions [a] to [d]

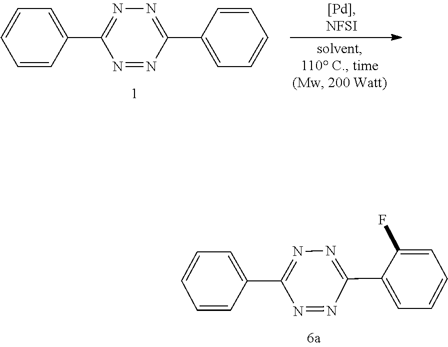

| | | NFSI | | | | |
|---|---|---|---|---|---|---|
| Entry | [Pd] | (equiv) | Solvent | Time | | 6a |
| 1 | PdCl2 | 1.0 | CH₃NO₂ | 17 | h | 34[b] |
| 2 | [PdCl(allyl)]₂ | 1.0 | CH₃NO₂ | 17 | h | 45[b] |
| 3 | Pd(dba)₂ | 1.0 | CH₃NO₂ | 17 | h | 41(30) |
| 4 | Pd2(dba)₃ | 1.0 | CH₃NO₂ | 17 | h | 35 |
| 5 | Pd2(dba)₃ | 1.0 | CH₃NO₂ | 17 | h | 46 |
| 6 | Pd2(dba)₃ | 1.5 | CH₃NO₂ | 17 | h | 57 |
| 7 | Pd2(dba)₃ | 1.0 | PhCF₃ | 17 | h | 47 |
| 8 | Pd(dba)₂ | 1.5 | PhCF₃ | 30 | min | 62 |
| 9 | Pd(dba)₂ | 2.0 | PhCF₃ | 10 | min | 71(44) |
| 10 | Pd(dba)₂ | 2.2 | PhCF₃ | 10 | min | 70(50) |
| 11 | Pd(dba)₂ | 2.5 | PhCF₃ | 10 | min | 63(47) |

[a] Conditions: 3,6-diphenyl-1,2,4,5-tetrazine (1) (1 equiv), [Pd] (10 mol %), NFSI (1-2.5 equiv), solvent (0.125M), 110° C., under argon. ¹H and ¹⁹F NMR yield and isolated yield under bracket.
[b]Chlorination product was detected.
[c] Pd₂(dba)₃ (20 mol %).
[d] Pd₂(dba)₃ (20 mol %), microwave 200 W, under air.

The first reaction between 3,6-diphenyl-1,2,4,5-tetrazine (1), and 1 equiv of N-fluorobenzenesulfonimide (NFSI) was conducted in nitromethane at 110° C., using 10 mol % of [Pd(dba)₂]. The monofluorinated compound (6a) was isolated in 30% yield, with a corresponding conversion in (1) around 41% over 17 h (entries 1-7). The reaction time may be dropped to 30 min, upon using microwave irradiation (entries 8-11), 20 mol % of [Pd(dba)₂], trifluoromethylbenzene (PhCF₃) as a solvent at 110° C. in the presence of air (entry 8). The amount of NFSI was crucial to achieve full conversion of (1). [9] Using 2.5 equiv of NFSI, (1) was fully converted into mono and difluorinated species (6a), (6b), (6c) in a [63:27:9] ratio (entry 11). A [77:18:5] ratio with a 91% conversion yield in (1) and 50% yield in isolated (6a) could be achieved using 2.2 equiv of NFSI (entry 10).

### A.4 Tetrafluorination of 3,6-diphenyl-1,2,4,5-tetrazine (1) or 3,6-bis(2-fluorophenyl)-1,2,4,5-tetrazine (6b)

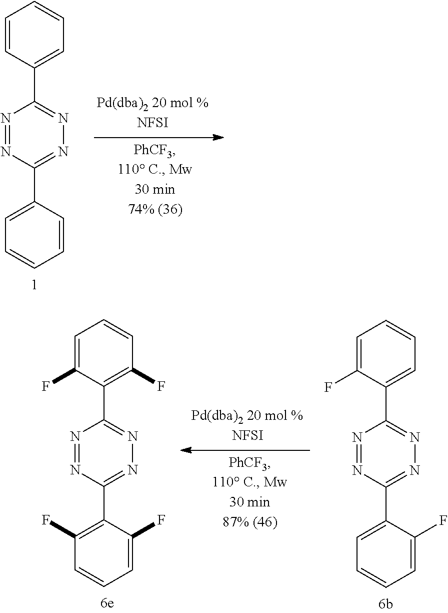

The synthesis of species (6e) may be of interest for future radiolabeled products incorporating four times more isotopes than (6a). This was achieved starting from either aryltetrazine (1) or its difluorinated derivative (6b).

### B—General Synthesis of Diphenyltetrazine

### 3,6-diphenyl-1,2,4,5-tetrazine (1): CAS 6830-78-0

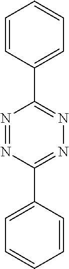 [SMILES: c1ccc(-c2nnc(-c3ccccc3)nn2)cc1]

To a mixture of benzonitrile (1 mL, 9.70 mmol) and hydrazine monohydrate (2.4 mL, 48.50 mmol) in absolute ethanol (10 mL) was added Sulfur (311 mg, 9.70 mmol). The resulting suspension was placed under nitrogen atmosphere, magnetically stirred and heated at 60° C. for 3 h. Upon cooling, the solvent was removed under reduced pressure to afford a yellowish solid. The crude mixture was dissolved in dichloromethane (2.9 mL), a solution of was NaNO₂ added (195, mL, 0.3 mM in distilled water), followed by addition of acetic acid (2.8 mL) at 0° C. A pink color develops that is characteristic of the tetrazine (I_{abs}=550 nm). The solvent was removed in vacuo and the crude product was purified by silica gel column chromatography (Dichloromethane-Heptane=1:1) to afford (1) (purple solid) in 30% (348.4 mg) yield.

¹H NMR (300 MHz, CDCl₃): δ (ppm)=8.68-8.66 (m, 4H), 7.67-7.61 (m, 6H).

### 3,6-bis(2-fluorophenyl)-1,2,4,5-tetrazine (6b): CAS 108350-48-7

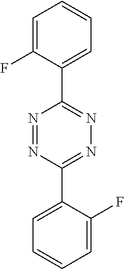 [SMILES: Fc1ccccc1-c1nnc(-c2ccccc2F)nn1]

To a mixture of 2-fluorobenzonitrile (0.88 mL, 8.26 mmol) and hydrazine monohydrate (2 mL, 41.30 mmol) in absolute ethanol (10 mL) was added sulfur (265 mg, 8.26 mmol). The resulting suspension was placed under nitrogen atmosphere, magnetically stirred and heated at 60° C. for 4 h. Upon cooling, the solvent was removed under reduced pressure to afford a yellowish solid. The crude mixture was dissolved in dichloromethane (2.5 mL), a solution of was NaNO₂ added (166 mL, 0.3 mM in distilled water), followed by addition of acetic acid (2.4 mL) at 0° C. A pink color develops that is characteristic of the tetrazine (I_{abs}=540 nm). The solvent was removed in vacuo and the crude product was purified by silica gel column chromatography (Dichloromethane-Heptane=1:1) to afford (1) (purple solid) in 10% (107.8 mg) yield.

¹H NMR (300 MHz, CDCl₃): δ (ppm)=8.38 (td, J=7.64, 1.77 Hz, 2H), 7.67-7.60 (m, 2H), 7.41 (td, J=7.74, 1.06 Hz, 2H), 7.34 (ddd, J=10.85, 8.34, 0.94 Hz, 2H); ¹⁹F NMR (282 MHz, CDCl₃): δ (ppm)=−111.6; ¹³C NMR (75 MHz, CDCl₃): δ (ppm)=163.4 (d, J=260.1 Hz), 163.2 (d, J=5.6 Hz), 134.3 (d, J=8.8 Hz), 131.5 (d, J=0.8 Hz), 124.9 (d, J=3.9 Hz), 120.6 (d, J=9.8 Hz), 117.6 (d, J=21.5 Hz); Elemental analysis: Calcd (%) for C₁₄H₈F₂N₄: C, 62.22, H, 2.98, N, 20.73. Found: C, 61.10, H, 2.84, N, 20.77; HRMS+p ESI (m/z) [M+Na⁺] Calcd for C₁₄H₈F₂N₄: 293.061. Found: m/z=293.060.

### C—General Procedure of Functionalization of Tetrazine

### 3-(2-bromophenyl)-6-phenyl-1,2,4,5-tetrazine (2a)

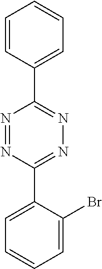 [SMILES: Brc1ccccc1-c1nnc(-c2ccccc2)nn1]

The 3,6-diphenyl-1,2,4,5-tetrazine (58.0 mg, 0.25 mmol), NBS (44.4 mg, 0.25 mmol), and Pd(dba)₂ (14.4 mg, 0.025 mmol) were introduced in a Schlenk tube, equipped with a magnetic stirring bar. 1,2-dichloroethane (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the brominated product. Then, the crude product was purified by silica gel column chromatography

(Dichloromethane-Heptane=1:1) to afford (2a) (purple solid) in 45% (34.9 mg) yield.

¹H NMR (300 MHz, CDCl₃): δ (ppm)=8.73-8.69 (m, 2H), 8.02 (ddd, J=7.49, 1.93, 0.23 Hz, 1H), 7.82 (ddd, J=7.94, 0.99, 0.30 Hz, 1H), 7.70-7.60 (m, 3H), 7.57 (td, J=8.06, 0.52 Hz, 1H), 7.47 (ddd, J=7.96, 7.54, 1.80 Hz, 1H); ¹³C NMR (75 MHz, CDCl₃): δ (ppm)=166.6, 163.3, 134.5, 133.9, 133.2, 132.5, 132.2, 131.7, 129.5, 128.6, 128.0, 122.5; Elemental analysis: Calcd (%) for C₁₄H₉BrN₄: C, 53.70, H, 2.90, N, 17.89. Found: C, 53.86, H, 2.73, N, 17.87; HRMS+p ESI (m/z) [M+H⁺] Calcd for C₁₄H₉BrN₄: 313.008. Found: m/z=313.008.

### 3,6-bis(2-bromophenyl)-1,2,4,5-tetrazine (2b): CAS 108350-48-7

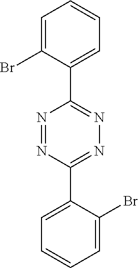 [SMILES: Brc1ccccc1-c1nnc(-c2ccccc2Br)nn1]

The 3,6-diphenyl-1,2,4,5-tetrazine (58.0 mg, 0.25 mmol), NBS (177.9 mg, 1.00 mmol), and Pd(dba)₂ (14.4 mg, 0.025 mmol) were introduced in a Schlenk tube, equipped with a magnetic stirring bar. 1,2-dichloroethane (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the brominated product.

Then, the crude product was filtered through a plug of silica (Dichloromethane-Heptane=1:1) to afford (2b) (purple solid) in 19% (18.6 mg) yield.

¹H NMR (300 MHz, CDCl₃): δ (ppm)=8.07 (dd, J=7.68, 1.76 Hz, 2H), 7.84 (dd, J=7.96, 1.13 Hz, 2H), 7.58 (td, J=7.52, 1.23 Hz, 2H), 7.49 (td, J=7.90, 1.81 Hz, 2H); ¹³C NMR (75 MHz, CDCl₃): δ (ppm)=165.7, 134.6, 133.6, 132.8, 132.5, 128.1, 122.7; Elemental analysis: Calcd (%) for C₁₄H₈Br₂N₄: C, 42.89, H, 2.06, N, 14.29. Found: C, 44.92, H, 2.66, N, 13.55; HRMS+p ESI (m/z) [M+H⁺] Calcd for C₁₄H₈Br₂N₄: 390.918. Found: m/z=390.919.

### 3-(2,6-dibromophenyl)-6-phenyl-1,2,4,5-tetrazine (2c)

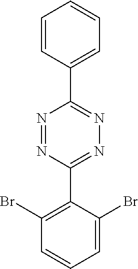 [SMILES: Brc1cccc(Br)c1-c1nnc(-c2ccccc2)nn1]

The 3,6-diphenyl-1,2,4,5-tetrazine (58.0 mg, 0.25 mmol), NBS (133.5 mg, 0.75 mmol), and Pd(dba)₂ (14.4 mg, 0.025 mmol) were introduced in a Schlenk tube, equipped with a magnetic stirring bar. 1,2-dichloroethane (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the brominated product. Then, the crude product was filtered through a plug of silica (Dichloromethane-Heptane=1:1) to afford (2c) (purple solid) in 14% (14.1 mg) yield.

¹H NMR (300 MHz, CDCl₃): δ (ppm)=8.77-8.73 (m, 2H), 7.76 (d, J=8.10 Hz, 2H), 7.69-7.62 (m, 3H), 7.33 (t, J=7.94 Hz, 1H); ¹³C NMR (75 MHz, CDCl₃): δ (ppm)=167.9, 163.9, 136.0, 133.4, 132.8, 132.3, 131.6, 129.6, 128.8, 124.1.

### 3-(2,6-dibromophenyl)-6-(2-bromophenyl)-1,2,4,5-tetrazine (2d)

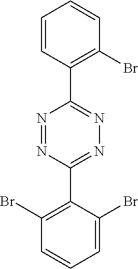 [SMILES: Brc1ccccc1-c1nnc(-c2c(Br)cccc2Br)nn1]

The 3,6-diphenyl-1,2,4,5-tetrazine (58.0 mg, 0.25 mmol), NBS (177.9 mg, 1.00 mmol), and Pd(dba)₂ (14.4 mg, 0.025 mmol) were introduced in a Schlenk tube, equipped with a magnetic stirring bar. 1,2-dichloroethane (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the brominated product. Then, the crude product was filtered through a plug of silica (Dichloromethane-Heptane=1:1) to afford (2d) (red solid) in 57% (66.8 mg) yield.

¹H NMR (300 MHz, CDCl₃): δ (ppm)=8.09 (ddd, J=7.72, 1.68, 0.11 Hz, 1H), 7.85 (ddd, J=7.92, 1.27, 0.28 Hz, 1H), 7.77 (d, J=8.10 Hz, 2H), 7.60 (td, J=7.52, 1.24 Hz, 1H), 7.53-7.47 (m, 1H), 7.35 (dd, J=8.33, 7.89 Hz, 1H); ¹³C NMR (75 MHz, CDCl₃): δ (ppm)=166.9, 166.4, 135.8, 134.5, 133.7, 133.0, 132.9, 132.4, 132.2, 128.1, 123.9, 122.7; Elemental analysis: Calcd (%) for C₁₄H₇Br₃N₄: C, 35.70, H, 1.50, N, 11.90. Found: C, 35.38, H, 1.16, N, 11.53; HRMS+p ESI (m/z) [M+H⁺] Calcd for C₁₄H₇Br₃N₄: 469.837. Found: m/z=469.837.

### 3,6-bis(2,6-dibromophenyl)-1,2,4,5-tetrazine (2e)

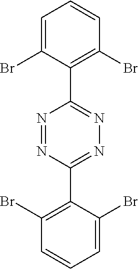 [SMILES: Brc1cccc(Br)c1-c1nnc(-c2c(Br)cccc2Br)nn1]

The 3,6-diphenyl-1,2,4,5-tetrazine (58.0 mg, 0.25 mmol), NBS (266.9 mg, 1.50 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the brominated product. Then, the crude product was filtered through a plug of silica (Dichloromethane-Heptane=1:1) to afford (2e) (pink solid) in 89% (122.8 mg) yield.

¹H NMR (300 MHz, CDCl₃): δ (ppm)=7.77 (d, J=8.10 Hz, 4H), 7.36 (t, J=8.20 Hz, 2H); ¹³C NMR (75 MHz, CDCl₃): δ (ppm)=167.6, 135.9, 133.1, 132.2, 123.7; Elemental analysis: Calcd (%) for C₁₄H₆Br₄N₄: C, 30.58, H, 1.10, N, 10.19. Found: C, 29.96, H, 1.38, N, 9.30; HRMS+p ESI (m/z) [M+Na⁺] Calcd for C₁₄H₆Br₄N₄: 568.721. Found: m/z=568.720.

### 3-(2-iodophenyl)-6-phenyl-1,2,4,5-tetrazine (3a)

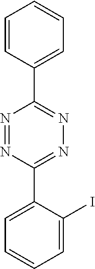 [SMILES: Ic1ccccc1-c1nnc(-c2ccccc2)nn1]

The 3,6-diphenyl-1,2,4,5-tetrazine (58.0 mg, 0.25 mmol), NIS (56.4 mg, 0.25 mmol), and Pd(dba)₂ (14.4 mg, 0.025 mmol) were introduced in a Schlenk tube, equipped with a magnetic stirring bar. 1,2-dichloroethane (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the iodinated product. Then, the crude product was purified by silica gel column chromatography (Dichloromethane-Heptane=1:1) to afford (3a) (purple solid) in 33% (29.8 mg) yield.

¹H NMR (300 MHz, CDCl₃): δ (ppm)=8.74-8.70 (m, 2H), 8.12 (dd, J=7.97, 0.95 Hz, 1H), 7.99 (dd, J=7.74, 1.60 Hz, 1H), 7.70-7.58 (m, 4H), 7.31-7.26 (m, 1H); ¹³C NMR (125 MHz, CDCl₃): δ (ppm)=167.5, 163.4, 141.2, 137.2, 133.2, 132.4, 131.7, 131.6, 129.5, 128.8, 128.6, 95.7; Elemental analysis: Calcd (%) for C₁₄H₉IN₄: C, 46.69, H, 2.52, N, 15.56. Found: C, 46.78, H, 2.22, N, 14.72; HRMS+p ESI (m/z) [M+Na⁺] Calcd for C₁₄H₉IN₄: 382.976. Found: m/z=382.975.

### 3,6-bis(2-iodophenyl)-1,2,4,5-tetrazine (3b)

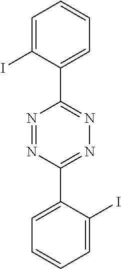 [SMILES: Ic1ccccc1-c1nnc(-c2ccccc2I)nn1]

The 3,6-diphenyl-1,2,4,5-tetrazine (58.0 mg, 0.25 mmol), NIS (140.6 mg, 0.63 mmol), and Pd(dba)₂ (14.4 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the iodinated product. Then, the crude product was purified by silica gel column chromatography (Dichloromethane-Heptane=1:1) to afford (3b) (purple solid) in 24% (29.5 mg) yield.

¹H NMR (300 MHz, CDCl₃): δ (ppm)=8.12 (dd, J=7.99, 0.93 Hz, 2H), 8.07 (dd, J=7.75, 1.61 Hz, 2H), 7.62 (td, J=7.58, 1.15 Hz, 2H), 7.33-7.28 (m, 2H); ¹³C NMR (75 MHz, CDCl₃): δ (ppm)=166.5, 141.2, 136.9, 132.6, 131.7, 128.8, 95.9; Elemental analysis: Calcd (%) for C₁₄H₈I₂N₄: C, 34.60, H, 1.66, N, 11.53. Found: C, 35.03, H, 1.22, N, 11.17; HRMS+p ESI (m/z) [M+Na⁺] Calcd for C₁₄H₈I₂N₄: 508.873. Found: m/z=508.872.

### 3-(2,6-diiodophenyl)-6-phenyl-1,2,4,5-tetrazine (3c)

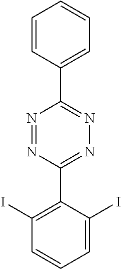 [SMILES: Ic1cccc(I)c1-c1nnc(-c2ccccc2)nn1]

The 3,6-diphenyl-1,2,4,5-tetrazine (58.0 mg, 0.25 mmol), NIS (140.6 mg, 0.63 mmol), and Pd(dba)₂ (14.4 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the iodinated product. Then, the crude product was purified by silica gel column chromatography (Dichloromethane-Heptane=1:1) to afford (3c) (purple solid) in 17% (21 mg) yield.

¹H NMR (300 MHz, CDCl₃): δ (ppm)=8.78-8.75 (m, 2H), 8.04 (d, J=7.97 Hz, 2H), 7.68-7.62 (m, 3H), 6.96 (t, J=7.96 Hz, 1H); ¹³C NMR (125 MHz, CDCl₃): δ (ppm)=171.4, 163.7, 142.7, 139.3, 133.4, 133.2, 131.6, 129.6, 129.5, 128.8, 128.2, 96.6; Elemental analysis: Calcd (%) for C₁₄H₉I₂N₄: C, 34.60, H, 1.66, N, 11.53. Found: C, 34.95, H, 2.52, N, 10.46; HRMS+p ESI (m/z) [M+Na⁺] Calcd for C₁₄H₉I₂N₄: 508.873. Found: m/z=508.872.

### 3-(2,6-diiodophenyl)-6-(2-iodophenyl)-1,2,4,5-tetrazine (3d)

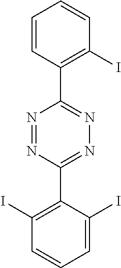 [SMILES: Ic1ccccc1-c1nnc(-c2c(I)cccc2I)nn1]

The 3,6-diphenyl-1,2,4,5-tetrazine (58.0 mg, 0.25 mmol), NIS (281.2 mg, 1.25 mmol), and Pd(dba)₂ (14.4 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the iodinated product. Then, the crude product was purified by silica gel column chromatography (Dichloromethane-Heptane=1:1) to afford (3d) (purple solid) in 20% (30.1 mg) yield.

¹H NMR (300 MHz, CDCl₃): δ (ppm)=8.14 (dd, J=7.88, 0.96 Hz, 1H), 8.13 (dd, J=7.75, 1.71 Hz, 1H), 8.05 (d, J=7.97 Hz, 2H), 7.64 (td, J=7.58, 1.14 Hz, 1H), 7.32 (td, J=7.63, 1.66 Hz, 1H), 6.98 (t, J=7.96 Hz, 1H); ¹³C NMR (75 MHz, CDCl₃): δ (ppm)=170.2, 166.9, 142.6, 141.2, 139.3, 137.0, 133.3, 132.8, 131.8, 128.9, 96.3, 96.0; Elemental analysis: Calcd (%) for C₁₄H₇I₃N₄: C, 27.48, H, 1.15, N, 9.16. Found: C, 28.14, H, 1.26, N, 8.72; HRMS+p ESI (m/z) [M+Na⁺] Calcd for C₁₄H₇I₃N₄: 634.769. Found: m/z=634.770.

### 3,6-bis(2,6-diiodophenyl)-1,2,4,5-tetrazine (3e)

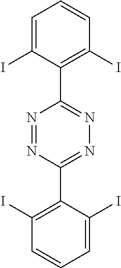 [SMILES: Ic1cccc(I)c1-c1nnc(-c2c(I)cccc2I)nn1]

The 3,6-diphenyl-1,2,4,5-tetrazine (58.0 mg, 0.25 mmol), NIS (674.9 mg, 3.00 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the iodinated product. Then, the crude product was purified by silica gel column chromatography (Ethyl acetate-Heptane=1:4, and then Dichloromethane=100%) to afford (3e) (pink solid) in 64% (118.1 mg) yield.

¹H NMR (300 MHz, CDCl₃): δ (ppm)=8.06 (d, J=7.97 Hz, 4H), 6.99 (t, J=7.96 Hz, 2H); ¹³C NMR (125 MHz, (CD₃)₂SO): δ (ppm)=170.7, 141.8, 138.8, 134.2, 97.2; Elemental analysis: Calcd (%) for C₁₄H₆I₄N₄: C, 22.79, H, 0.82, N, 7.59. Found: C, 23.26, H, 0.73, N, 7.27; HRMS+p ESI (m/z) [M+H⁺] Calcd for C₁₄H₆I₄N₄: 738.684. Found: m/z=738.684.

### 3-(2-chlorophenyl)-6-phenyl-1,2,4,5-tetrazine (4a): CAS74115-26-7

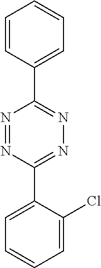 [SMILES: Clc1ccccc1-c1nnc(-c2ccccc2)nn1]

The 3,6-diphenyl-1,2,4,5-tetrazine (58.0 mg, 0.25 mmol), NCS (44.4 mg, 0.25 mmol), and PdCl₂ (4.4 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the chlorinated product. Then, the crude product was purified by silica gel column chromatography (Dichloromethane-Heptane=1:1) to afford (4a) (purple solid) in 32% (21.3 mg) yield.

¹H NMR (300 MHz, CDCl₃): δ (ppm)=8.72-8.69 (m, 2H), 8.08-8.05 (m, 1H), 7.70-7.60 (m, 4H), 7.56-7.49 (m, 2H); ¹³C NMR (75 MHz, CDCl₃): δ (ppm)=165.9, 163.3, 133.9, 133.2, 132.5, 132.2, 131.9, 131.7, 131.3, 129.5, 128.5, 127.5; HRMS+p ESI (m/z) [M+H⁺] Calcd for C₁₄H₉ClN₄: 269.058. Found: m/z=269.058.

### 3,6-bis(2-chlorophenyl)-1,2,4,5-tetrazine (4b): CAS 74115-24-5

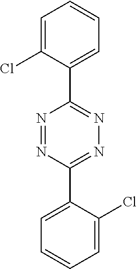 [SMILES: Clc1ccccc1-c1nnc(-c2ccccc2Cl)nn1]

The 3,6-diphenyl-1,2,4,5-tetrazine (58.0 mg, 0.25 mmol), NCS (166.9 mg, 1.25 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the chlorinated product. Then, the crude product was purified by silica gel column chromatography (Dichloromethane-Heptane=1:1) to afford (4b) (purple solid) in 35% (29.4 mg) yield.

¹H NMR (300 MHz, CDCl₃): δ (ppm)=8.13-8.10 (m, 2H), 7.66-7.63 (m, 2H), 7.60-7.50 (m, 4H); ¹³C NMR (75 MHz, CDCl₃): δ (ppm)=165.1, 134.0, 132.8, 132.5, 131.7, 131.4, 127.75; HRMS+p ESI (m/z) [M+H⁺] Calcd for C₁₄H₈C₁₂N₄: 303.019. Found: m/z=303.019.

### 3-(2,6-dichlorophenyl)-6-(2-chlorophenyl)-1,2,4,5-tetrazine (4d)

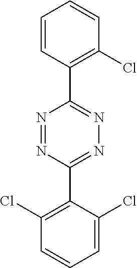 [SMILES: Clc1ccccc1-c1nnc(-c2c(Cl)cccc2Cl)nn1]

The 3,6-diphenyl-1,2,4,5-tetrazine (58.0 mg, 0.25 mmol), NCS (200.3 mg, 1.50 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the chlorinated product. Then, the crude product was purified by silica gel column chromatography (Dichloromethane-Heptane=1:1) to afford (4d) (purple solid) in 8% (6.8 mg) yield.

¹H NMR (300 MHz, CDCl₃): δ (ppm)=8.15-8.11 (m, 1H), 7.67-7.61 (m, 1H), 7.59-7.49 (m, 5H); ¹³C NMR (125 MHz, CDCl₃): δ (ppm)=165.8, 164.9, 135.4, 134.1, 132.9, 132.5, 132.4, 132.3, 131.7, 131.4, 128.6, 127.5.

### 3,6-bis(2,6-dichlorophenyl)-1,2,4,5-tetrazine (4e): CAS 162320-76-5

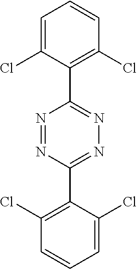 [SMILES: Clc1cccc(Cl)c1-c1nnc(-c2c(Cl)cccc2Cl)nn1]

The 3,6-diphenyl-1,2,4,5-tetrazine (58.0 mg, 0.25 mmol), NCS (333.8 mg, 2.50 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the chlorinated product. Then, the crude product was purified by silica gel column chromatography (Dichloromethane-Heptane=1:1) to afford (4e) (pink solid) in 34% (31.7 mg) yield.

¹H NMR (300 MHz, CDCl₃): δ (ppm)=7.58-7.48 (m, 6H); ¹³C NMR (75 MHz, CDCl₃): δ (ppm)=165.5, 135.2, 132.5, 132.3, 128.6; HRMS+p ESI (m/z) [M+H⁺] Calcd for C₁₄H₆C₁₄N₄: 370.941. Found: m/z=370.943.

### 3-(2-bromophenyl)-6-phenyl-1,2,4,5-tetrazine (5a)

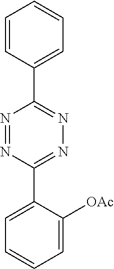 [SMILES: CC(=O)Oc1ccccc1-c1nnc(-c2ccccc2)nn1]

The 3,6-diphenyl-1,2,4,5-tetrazine (58.0 mg, 0.25 mmol), PhI(OAc)₂ (80.5 mg, 0.25 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water. The combined organic layer was washed with water and dried over MgSO₄. The solvent was removed in vacuo and the residue was analyzed by NMR to determine the conversion of the acetylated product. Then, the crude product was purified by silica gel column chromatography (Dichloromethane=100%) to afford (5a) (purple solid) in 43% (31.4 mg) yield.

¹H NMR (300 MHz, CDCl₃): δ (ppm)=8.68-8.64 (m, 2H), 8.49 (dd, J=7.87, 1.70 Hz, 1H), 7.69-7.58 (m, 4H), 7.51 (td, J=7.77, 1.24 Hz, 1H), 7.30 (dd, J=8.09, 1.15 Hz, 1H), 2.40 (s, 3H); ¹³C NMR (75 MHz, CDCl₃): δ (ppm)=170.1, 164.1, 163.3, 149.9, 133.4, 132.9, 131.8, 131.2, 129.4, 128.3, 126.9, 125.2, 124.7, 21.2; Elemental analysis: Calcd (%) for C₁₆H₁₂N₄O₂: C, 65.75, H, 4.14, N, 19.17. Found: C, 65.04, H, 4.32, N, 18.78.

HRMS+p ESI (m/z) [M+Na⁺] Calcd for C₁₆H₁₂N₄O₂: 315.085. Found: m/z=315.084.

### 3,6-bis(2-acetoxyphenyl)-1,2,4,5-tetrazine (5b)

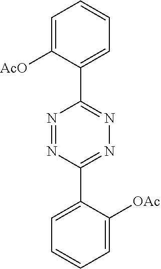 [SMILES: CC(=O)Oc1ccccc1-c1nnc(-c2ccccc2OC(C)=O)nn1]

The 3,6-diphenyl-1,2,4,5-tetrazine (58.0 mg, 0.25 mmol), PhI(OAc)₂ (241.6 mg, 0.75 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water. The combined organic layer was washed with water and dried over MgSO₄. The solvent was removed in vacuo and the residue was analyzed by NMR to determine the conversion of the acetylated product. Then, the crude product was purified by silica gel column chromatography (Dichloromethane=100%) to afford (5b) (red solid) in 29% (25.4 mg) yield.

¹H NMR (300 MHz, CDCl₃): δ (ppm)=8.50 (dd, J=7.88, 1.66 Hz, 2H), 7.67 (ddd, J=9.19, 7.49, 1.72 Hz, 2H), 7.52 (td, J=7.79, 1.24 Hz, 2H), 7.30 (dd, J=8.10, 1.10 Hz, 2H), 2.37 (s, 6H); ¹³C NMR (75 MHz, CDCl₃): δ (ppm)=170.0, 163.2, 150.0, 133.7, 131.4, 126.9, 125.0, 124.8, 21.2; Elemental analysis: Calcd (%) for C₁₈H₁₄N₄O₄: C, 61.71, H, 4.03, N, 15.99. Found: C 61.19.11, H, 4.32, N, 15.34; HRMS+p ESI (m/z) [M+Na⁺] Calcd for C₁₆H₁₂N₄O₂: 373.090. Found: m/z=373.089.

### 3-(2,6-diacetoxyphenyl)-6-phenyl-1,2,4,5-tetrazine (5c)

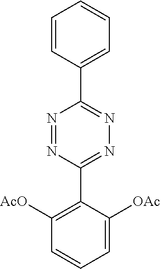 [SMILES: CC(=O)Oc1cccc(OC(C)=O)c1-c1nnc(-c2ccccc2)nn1]

The 3,6-diphenyl-1,2,4,5-tetrazine (58.0 mg, 0.25 mmol), PhI(OAc)₂ (241.6 mg, 0.75 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 100° C. and reactants were allowed to stir for 17 h. After cooling to room temperature, the reaction mixture was diluted with dichloromethane, and was washed three times with water. The combined organic layer was washed with water and dried over MgSO₄. The solvent was removed in vacuo and the residue was analyzed by NMR to determine the conversion of the acetylated product. Then, the crude product was purified by silica gel column chromatography (Dichloromethane=100%) to afford (5c) (red solid) in 11% (9.6 mg) yield.

¹H NMR (300 MHz, CDCl₃): δ (ppm)=8.69-8.66 (m, 2H), 7.68-7.60 (m, 4H), 7.27 (d, J=8.26 Hz, 2H), 2.22 (s, 6H); ¹³C NMR (125 MHz, CDCl₃): δ (ppm)=169.2, 163.3, 163.0, 150.2, 133.2, 132.3, 131.8, 129.5, 128.6, 121.9, 120.0, 20.9; Elemental analysis: Calcd (%) for C₁₈H₁₄N₄O₄: C, 61.71, H, 4.03, N, 15.99. Found: C, 61.15, H, 4.26, N, 15.37.

HRMS+p ESI (m/z) [M+Na⁺] Calcd for C₁₆H₁₂N₄O₂: 373.090. Found: m/z=373.089.

### 3-(2,6-diacetoxyphenyl)-6-(2-acetoxyphenyl)-1,2,4,5-tetrazine (5d)

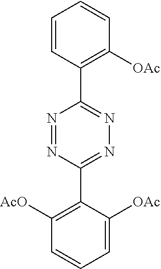 [SMILES: CC(=O)Oc1ccccc1-c1nnc(-c2c(OC(C)=O)cccc2OC(C)=O)nn1]

The 3,6-diphenyl-1,2,4,5-tetrazine (58.0 mg, 0.25 mmol), PhI(OAc)₂ (805.3 mg, 2.50 mmol), and Pd(OAc)₂ (5.6 mg, 0.025 mmol) were introduced in a Schlenk tube, equipped with a magnetic stirring bar. Acetic acid (2 mL) was added, and the Schlenk tube purged several times with argon. The Schlenk tube was placed in a pre-heated oil bath at 120° C. and reactants were allowed to stir for 17 h. After cooling to room temperature, the reaction mixture was diluted with dichloromethane, and was washed three times with water. The combined organic layer was washed with water and dried over MgSO₄. The solvent was removed in vacuo and the residue was analyzed by NMR to determine the conversion of the acetylated product. Then, the crude product was purified by silica gel column chromatography (Dichloromethane=100%) to afford (5d) (red solid) in 21% (21.4 mg) yield.

¹H NMR (300 MHz, CDCl₃): δ (ppm)=8.54 (dd, J=7.89, 1.67 Hz, 1H), 7.71-7.65 (m, 1H), 7.27 (t, J=8.26 Hz, 1H), 7.55-7.50 (m, 1H), 7.30 (dd, J=8.10, 1.10 Hz, 1H), 7.27 (d, J=8.26 Hz, 2H), 2.35 (s, 3H), 2.20 (s, 6H); ¹³C NMR (75 MHz, CDCl₃): δ (ppm)=170.0, 169.1, 163.1, 162.2, 150.2, 133.9, 132.4, 131.6, 127.0, 124.8, 121.9, 119.8, 21.1, 20.8; Elemental analysis: Calcd (%) for C₂₀H₁₆N₄O₆: C, 58.82, H, 3.95, N, 13.72. Found: C, 57.98, H, 3.66, N, 13.13; HRMS+p ESI (m/z) [M+Na⁺] Calcd for C₂₀H₁₆N₄O₆: 409.114. Found: m/z=409.114.

### 3-(2-fluorophenyl)-6-phenyl-1,2,4,5-tetrazine (6a)

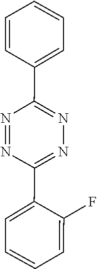 [SMILES: Fc1ccccc1-c1nnc(-c2ccccc2)nn1]

The 3,6-diphenyl-1,2,4,5-tetrazine (58.0 mg, 0.25 mmol), NFSI (78.8 mg, 0.25 mmol), and Pd(dba)₂ (14.4 mg, 0.025 mmol) were introduced in a Schlenk tube, equipped with a magnetic stirring bar. Nitromethane (2 mL) was added, and the Schlenk tube purged several times with argon. The Schlenk tube was placed in a pre-heated oil bath at 110° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the fluorinated product. Then, the crude product was purified by silica gel column chromatography (Dichloromethane-Heptane=1:1) to afford (6a) (purple solid) in 30% (18.7 mg) yield.

¹H NMR (300 MHz, CDCl₃): δ (ppm)=8.70-8.66 (m, 2H), 8.34 (td, J=7.63, 1.77 Hz, 1H), 7.68-7.58 (m, 4H), 7.40 (td, J=7.70, 1.14 Hz, 1H), 7.33 (ddd, J=10.87, 8.31, 0.96 Hz, 1H); ¹⁹F NMR (282 MHz, CDCl₃): δ (ppm)=−112.0;

¹³C NMR (75 MHz, CDCl₃): δ (ppm)=164.0 (d, J=5.9 Hz), 163.4 (d, J=5.6 Hz), 159.9, 134.1 (d, J=8.7 Hz), 133.0, 131.7, 131.4 (d, J=0.9 Hz), 129.5, 128.4, 124.9 (d, J=3.9 Hz), 120.9 (d, J=9.9 Hz), 117.7 (d, J=21.8 Hz); Elemental analysis: Calcd (%) for C₁₄H₉FN₄: C, 66.66, H, 3.60, N, 22.21. Found: C, 65.49, H, 3.53, N, 21.16; HRMS+p ESI (m/z) [M+H⁺] Calcd for C₁₄H₉FN₄: 253.088. Found: m/z=253.088.

### 3,6-bis(2-fluorophenyl)-1,2,4,5-tetrazine (6b): CAS 108350-48-7

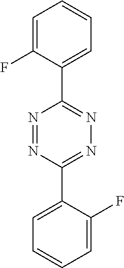 [SMILES: Fc1ccccc1-c1nnc(-c2ccccc2F)nn1]

The 3,6-diphenyl-1,2,4,5-tetrazine (58.0 mg, 0.25 mmol), NFSI (275.9 mg, 0.88 mmol), and Pd(dba)₂ (28.8 mg, 0.05 mmol) were introduced in a Schlenk tube, equipped with a magnetic stirring bar. Dry trifluoromethylbenzene (2 mL) was added, and the Schlenk tube purged several times with argon. The Schlenk tube was placed in a pre-heated oil bath at 110° C. and reactants were allowed to stir for 17 h. After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the fluorinated product. Then, the crude product was purified by silica gel column chromatography (Dichloromethane-Heptane=1:1) to afford (6b) (purple solid) in 30% (20.3 mg) yield.

¹H NMR (300 MHz, CDCl₃): δ (ppm)=8.38 (td, J=7.64, 1.77 Hz, 2H), 7.67-7.60 (m, 2H), 7.41 (td, J=7.74, 1.06 Hz, 2H), 7.34 (ddd, J=10.85, 8.34, 0.94 Hz, 2H); ¹⁹F NMR (282 MHz, CDCl₃): δ (ppm)=−111.6; ¹³C NMR (75 MHz, CDCl₃): δ (ppm)=163.4 (d, J=260.1 Hz), 163.2 (d, J=5.6 Hz), 134.3 (d, J=8.8 Hz), 131.5 (d, J=0.8 Hz), 124.9 (d, J=3.9 Hz), 120.6 (d, J=9.8 Hz), 117.6 (d, J=21.5 Hz); Elemental analysis: Calcd (%) for C₁₄H₈F₂N₄: C, 62.22, H, 2.98, N, 20.73. Found: C, 61.10, H, 2.84, N, 20.77; HRMS+p ESI (m/z) [M+Na⁺] Calcd for C₁₄H₈F₂N₄: 293.061. Found: m/z=293.060.

### 3,6-bis(2,6-difluorophenyl)-1,2,4,5-tetrazine (6e)

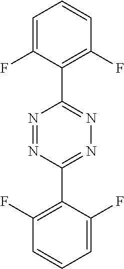 [SMILES: Fc1cccc(F)c1-c1nnc(-c2c(F)cccc2F)nn1]

The 3,6-bis(2-fluorophenyl)-1,2,4,5-tetrazine (67.5 mg, 0.25 mmol), NFSI (630.7 mg, 2 mmol), and Pd(dba)₂ (28.8 mg, 0.05 mmol) were introduced in a 10 mL microwave reaction vessel, equipped with a magnetic stirring bar. Dry trifluoromethylbenzene (2 mL) was added, and the reaction mixture was heated in the microwave at 110° C. for 30 min (200 W, 2 min ramp). After cooling to room temperature, the reaction mixture was diluted with dichloromethane, and was washed three times with water+3% of TEA. The combined organic layer was washed with water and dried over MgSO₄. The solvent was removed in vacuo and the residue was analyzed by NMR to determine the conversion of the fluorinated product. Then, the crude product was purified by silica gel column chromatography (Dichloromethane-Heptane=1:1, then dichloromethane=100%) to afford (6e) (red solid) in 46% (35.2 mg) yield.

¹H NMR (300 MHz, CDCl₃): δ (ppm)=7.66-7.57 (m, 2H), 7.22-7.14 (m, 4H); ¹⁹F NMR (282 MHz, CDCl₃): δ (ppm)=−112.4; ¹³C NMR (75 MHz, CDCl₃): δ (ppm)=163.0 (dd, J=5.5, 257.3 Hz), 161.5 (m), 134.0 (t, J=10.5 Hz), 112.7 (AA′X, N=12.5 Hz), 111.9 (t, J=16.8 Hz); Elemental analysis: Calcd (%) for C₁₄H₆F₄N₄: C, 54.91, H, 1.97, N, 18.30. Found: C, 54.61, H, 1.72, N, 18.34; HRMS+p ESI (m/z) [M+H⁺] Calcd for C₁₄H₆F₄N₄: 307.060. Found: m/z=307.060.

### D—Typical Procedure of Inverse Electron-Demand Diels-Alder Reaction

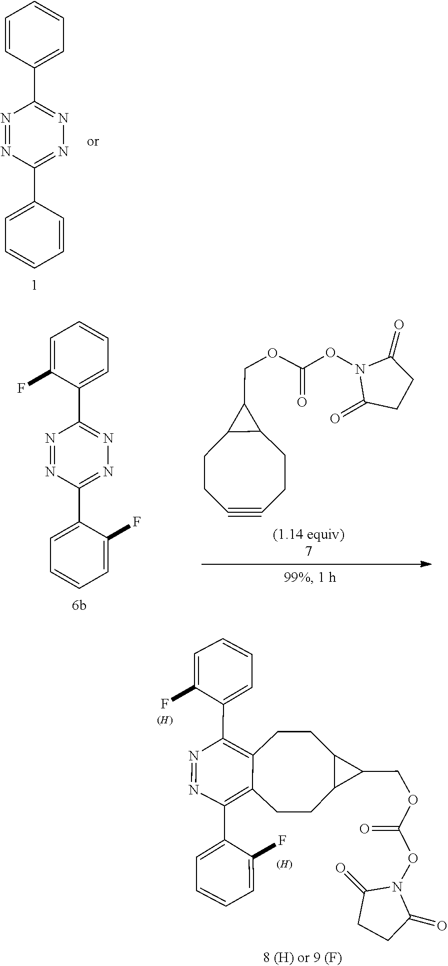

Strained-promoted [4+2] cycloaddition of 1,2,4,5-tetrazine ((1) or (6b)) with bicyclononyne was monitored by UV/Vis spectroscopy upon careful examination of the decay of the absorption band at 540 nm (6b) and 550 nm (1). The reaction went to completion in less than an hour with (6b), and was slightly longer with (1). Such a result shows that the presence of the F atoms did not prevent the cycloaddition, but it affects the electron-density of the dieneophile increasing the rate of the reaction.

The reaction was also done on the following compounds:

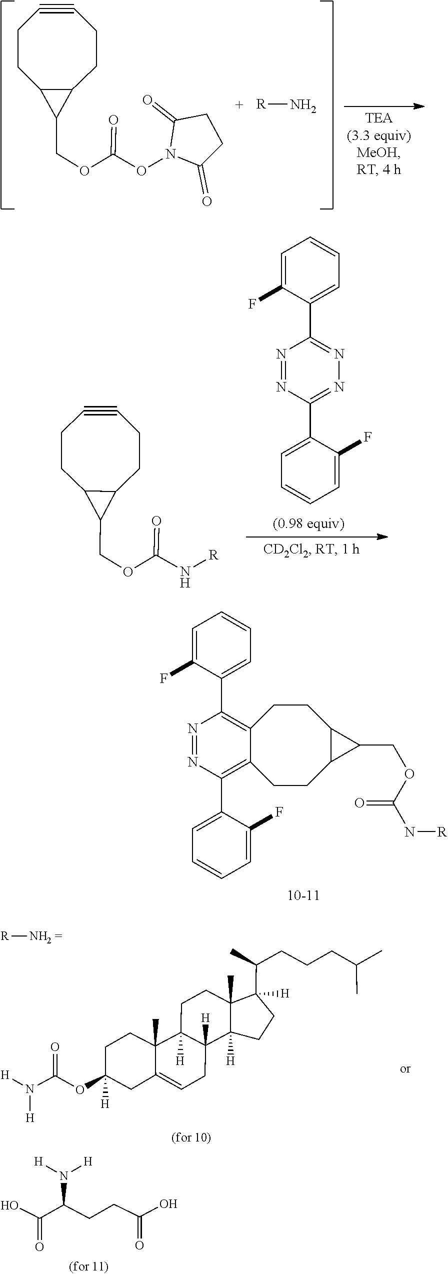

### Synthesis

A solution of 3,6-diphenyl-1,2,4,5-tetrazine with a concentration of 1 mM (2.3 mg into 10 mL of THF) and a solution of cyclooctyne with a concentration of 46 mM (2 mg into 150 μL of THF) were prepared. The 1 mM solution of 3,6-diphenyl-1,2,4,5-tetrazine (2 mL) and the 46 mM solution of cyclooctyne (50 μL) were mixed into a glass cuvette and the reaction was followed by UV/Vis spectrophotometer.

The procedure was the same with a solution of 3,6-bis(2-fluorophenyl)-1,2,4,5-tetrazine with a concentration of 1 mM (2.5 mg into 10 mL of THF) instead of 3,6-diphenyl-1,2,4,5-tetrazine.

### EXAMPLE 2: OPTIMIZATION OF THE SYNTHESIS OF UNEQUALLY HALOGENATED COMPOUNDS OF FORMULA (IA)

### Material

All reagents were purchased from commercial suppliers and used without purifications. All reactions were performed in Schlenk tubes or in a microwave reaction vessel. Microwave heating was carried out using a CEM Discover microwave reactor. The microwave reactions were run in closed reaction vessels with magnetic stirring and with the temperature controlled via IR detection. ¹H (300 MHz), 13C (75 or 125 MHz), ¹⁹F (282 MHz) spectra were recorded on Brucker AVANCE III instrument in CDCl₃ solutions. Chemical shifts are reported in ppm relative to CDCl3 (¹H: 7.26 and ¹³C: 77.16) and coupling constants J are given in Hz. High resolution mass spectra (HRMS) were obtained on a Thermo LTQ-Orbitrap XL with ESI source. Flash chromatography was performed on silica gel (230-400 mesh). Elemental analysis experiments were performed Thermo Electron Flash EA 1112 Series.

### Results:

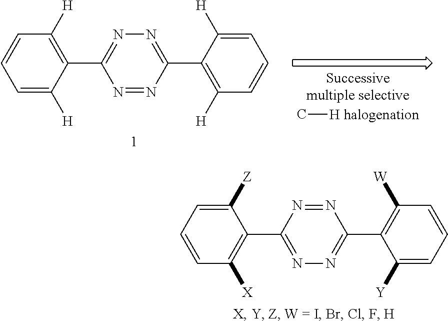

Fast synthetic access to halogenated s-aryltetrazines has been extended towards a wider set of functionalized compounds, including polyhalogenated molecules, including the symmetrical ortho-difunctionalized previously described 2b-6b.

### A.1 Optimisation of C—H Mono-Bromination of 3,6-diphenyl-1,2,4,5-tetrazine (1) Under Conditions [a] to [f]

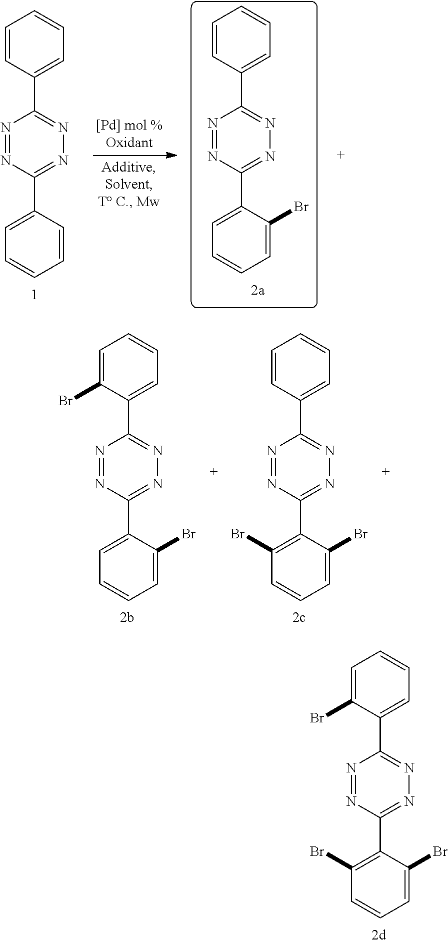

| | | Oxidant | | Conv. | 2a | 2b | 2c | 2d |
|---|---|---|---|---|---|---|---|---|
| Entry | [Pd] | (equiv) | Solvent | (%) | (%) | (%) | (%) | (%) |
| 1 | X | NBS (1.0) | CH₃NO₂ | 0 | 0 | 0 | 0 | 0 |
| 2^{[b]} | Pd(dba)₂ | NBS (1.0) | CH₃NO₂ | 44 | 41 | 3 | 0 | 0 |
| | | | | | (25) | | | |
| 3 | Pd(dba)₂ | NBS (1.0) | CH₃NO₂ | 53 | 47 | 7 | 0 | 0 |
| | | | | | (47) | | | |
| 4 | Pd₂(dba)₃ | NBS (1.0) | CH₃NO₂ | 51 | 46 | 5 | 0 | 0 |
| | | | | | (38) | | | |
| 5 | Pd(OAc)₂ | NBS (1.0) | CH₃NO₂ | 76 | 61 | 9 | 6 | 0 |
| | | | | | (52) | (6) | | |
| 6^{[c]} | Pd(dba)₂ | NBS (1.0) | CH₃NO₂ | 53 | 49 | 4 | 0 | 0 |
| | | | | | (30) | | | |
| 7 | Pd(dba)₂ | TBATB (1.0) | CH₃NO₂ | 0 | 0 | 0 | 0 | 0 |
| 8 | Pd(dba)₂ | PTB (1.0) | CH₃NO₂ | 0 | 0 | 0 | 0 | 0 |
| 9 | Pd(dba)₂ | NBS (2.0) | CH₃NO₂ | 69 | 53 | 9 | 7 | 0 |
| | | | | | (46) | | | |
| 10 | Pd(dba)₂ | NBS (3.0) | CH₃NO₂ | 87 | 54 | 22 | 8 | 3 |
| | | | | | (46) | (20) | | |
| 11 | Pd(dba)₂ | NBS (1.0) | PhCF₃ | 47 | 42 | 5 | 0 | 0 |
| 12^{[d]} | Pd(dba)₂ | NBS (1.0) | HOAc | 55 | 48 | 6 | 1 | 0 |
| 13^{[e]} | Pd(dba)₂ | NBS (1.0) | DCE | 26 | 25 | 1 | 0 | 0 |
| 14^{[f]} | Pd(dba)₂ | NBS (1.0) | CH₃NO₂ | 28 | 27 | 1 | 0 | 0 |
| 15^{[g]} | Pd(dba)₂ | NBS (1.0) | CH₃NO₂ | 60 | 49 | 8 | 3 | 0 |
| 16^{[d]} | Pd(OAc)₂ | NBS (1.0) | HOAc | 81 | 61 | 15 | 5 | 0 |
| | | | | | (53) | (13) | | |
| 17^{[d]} | Pd(OAc)₂ | NBS (2.0) | HOAc | 100 | 28 | 51 | 11 | 10 |
| | | | | | | (45) | | (5) |

^{[a]}Conditions: 3,6-diphenyl-1,2,4,5-tetrazine (1) (1 equiv), [Pd] (10 mol %), [X] (1-3.0 equiv), solvent (0.125 M), 100° C., microwave 200 W, under air, 10 min. ¹H NMR yield and isolated yield under bracket. dba: dibenzylidene acetone. TBATB = tetrabutylammonium tribromide. PTB = pyridinium tribromide. DCE: dichloroethane.
^{[b]}Under argon.
^{[c]}[Pd] (20 mol %).
^{[d]}110° C. instead of 100° C.
^{[e]}90° C. instead of 100° C.
^{[f]}80° C. instead of 100° C.
^{[g]}30 mn instead of 10 min.

In the absence of palladium no reaction occurred. For practical reasons catalytic conditions screening was achieved under air. It was successfully established that inert gas conditions are unnecessary for C—H activation/bromination. The reaction using 10 mol % of zerovalent palladium precursors Pd₂(dba)₃, and Pd(dba)₂, in nitromethane at 100° C., afforded the expected 3-(2-bromophenyl)-6-(phenyl)-1,2,4,5-tetrazine (2a) with only traces of dibrominated 3,6-bis(2-bromophenyl)-1,2,4,5-tetrazine (2b) (<5%). The palladium (II) precursor Pd(OAc)₂, known for its efficiency in sp²C—H activation, furnished a better conversion albeit with a slightly lower selectivity. Then compound (2a) can be easily purified and isolated in 52% yield. Other solvents, such as trifluoromethylbenzene, acetic acid or 1,2-dichlorethane led to lower conversions. In comparison, under thermal heating condition, the best catalytic system we determined for the production of (2a) furnished 45% isolated yield after 17 h under argon.

### A.2 Optimisation of Mono-Iodination of 3,6-diphenyl-1,2,4,5-tetrazine (1) Under Conditions [a] or [b]

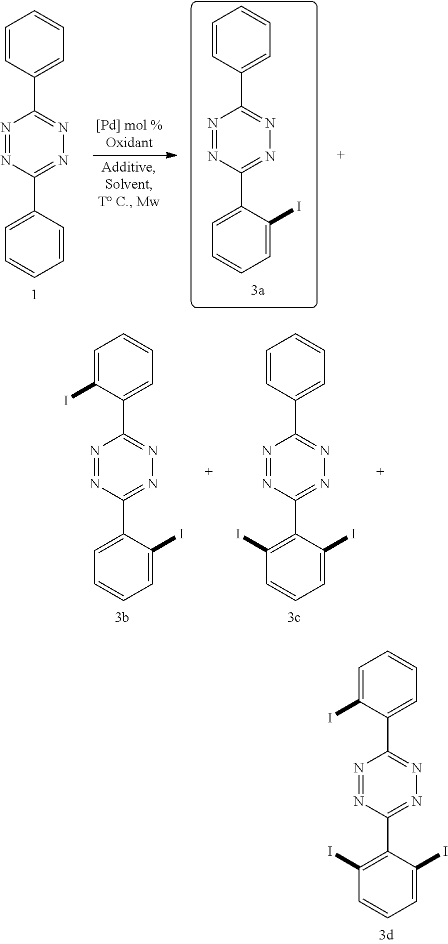

| | | Oxidant | | Conv. | 3a | 3b | 3c | 3d |
|---|---|---|---|---|---|---|---|---|
| Entry | [Pd] | (equiv) | Solvent | (%) | (%) | (%) | (%) | (%) |
| 1 | Pd(dba)₂ | NIS (1.0) | CH₃NO₂ | 10 | 10 | 0 | 0 | 0 |
| 2 | Pd(dba)₂ | NIS (1.0) | AcOH | 66 | 54 | 9 | 3 | 0 |
| | | | | | (47) | (4) | | |
| 3^{[b]} | Pd(OAc)₂ | NIS (1.0) | AcOH | 82 | 56 | 19 | 7 | 0 |
| | | | | | (34) | (11) | | |

^{[a]}Conditions: 3,6-diphenyl-1,2,4,5-tetrazine (1) (1 equiv), [Pd] (10 mol %), NIS (1 equiv), solvent (0.125 M), 100° C., microwave 200 W, under air, 10 min. ¹H NMR yield and isolated yield under bracket.
^{[b]}110° C. instead of 100° C.

With the microwave assisted protocol for monobromination, a fast and facile access to monoiodinated s-aryltetrazines using N-iodosuccinimide was achieved. The monoiodinated product (3a) was obtained in 47% isolated yield. The diiodinated aryltetrazine (3b) was obtained using 2 equiv of NIS with [Pd(OAc)₂] as the catalyst in AcOH in 10 min at 120° C. with 45% isolated yield. Side products of 3-(2,6-diiodophenyl)-6-phenyl-1,2,4,5-tetrazine (3c) (22%) and (3d) (27%).

### A.3 Optimisation of Mono-Chlorination of 3,6-diphenyl-1,2,4,5-tetrazine (1) Under Conditions [a] to [d]

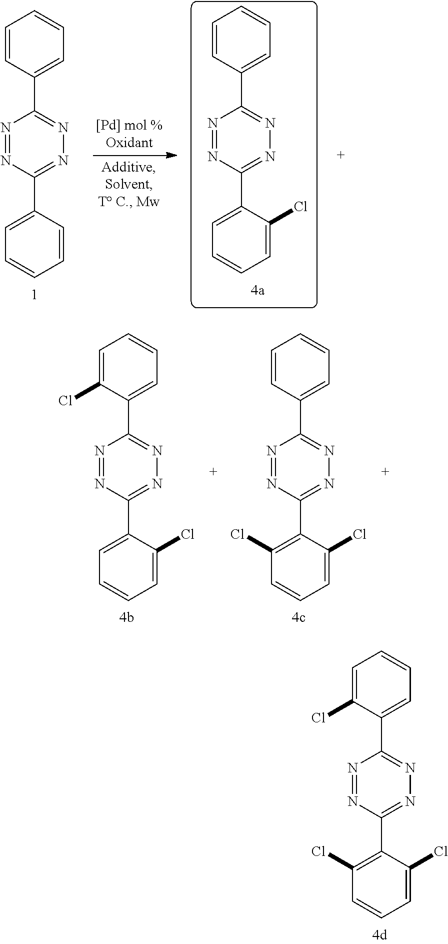

| | | Oxidant | | Conv. | 4a | 4b | 4c | 4d |
|---|---|---|---|---|---|---|---|---|
| Entry | [Pd] | (equiv) | Solvent | (%) | (%) | (%) | (%) | (%) |
| 1 | Pd(dba)₂ | NCS (1.0) | CH₃NO₂ | 0 | 0 | 0 | 0 | 0 |
| 2^{[b]} | Pd(dba)₂ | NCS (1.0) | AcOH | 47 | 43 | 4 | 0 | 0 |
| | | | | | (33) | | | |
| 3^{[b]} | Pd(OAc)₂ | NCS (1.0) | AcOH | 45 | 41 | 4 | 0 | 45 |
| | | | | | (37) | | | |
| 4^{[b]} | Pd(dba)₂ | NCS (3.0) | AcOH | 79 | 56 | 15 | 8 | 0 |
| | | | | | (54) | (9) | | |

^{[a]}Conditions: 3,6-diphenyl-1,2,4,5-tetrazine (1) (1 equiv), [Pd] (10 mol %), NCS (1-2.5 equiv), solvent (0.125 M), 100° C., microwave 200 W, under air, 45 min. ¹H NMR yield and isolated yield under bracket.
^{[b]}110° C. instead of 100° C.
^{[d]}PivOH (30% mol).

With the microwave assisted protocol for monobromination, a fast and facile access to monochlorinated s-aryltetrazines using the appropriate N-halosuccinimide was achieved. The monochlorinated product (4a) was obtained in 54% isolated yield.

The dichlorinated s-aryltetrazine (4b) was synthesized using of NCS with [Pd(OAc)₂] as the catalyst in AcOH 120° C. in only a few minutes and was isolated in 37% yield.

### A.4 Tetrabromination of 3,6-diphenyl-1,2,4,5-tetrazine (1) Under Conditions [a] to [f]

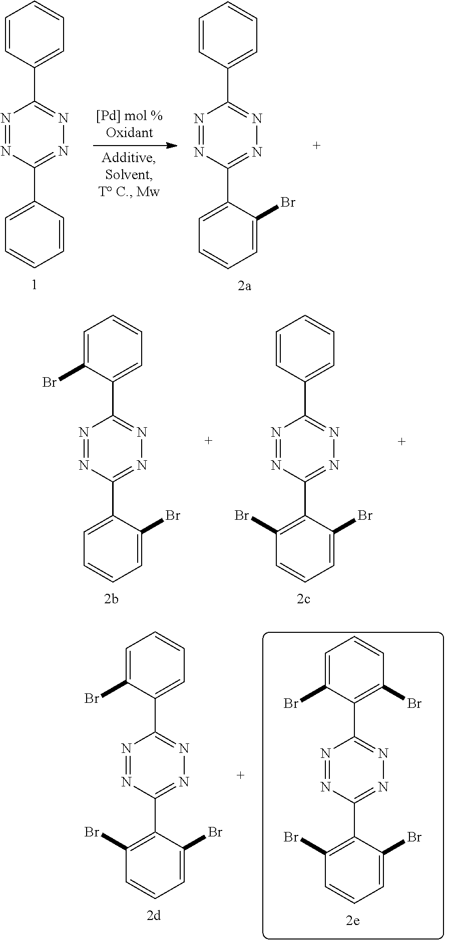

| | | Oxidant | | Conv. | 2a | 2b | 2c | 2d | 2e |
|---|---|---|---|---|---|---|---|---|---|
| Entry | [Pd] | (equiv) | Solvent | (%) | (%) | (%) | (%) | (%) | (%) |
| 1 | Pd(OAc)₂ | NBS (3.0) | AcOH | 100 | 0 | 43 | 6 | 48 | 3 |
| | | | | | | (37) | | (41) | |
| 2 | Pd(OAc)₂ | NBS (5.0) | AcOH | 100 | 0 | 0 | 0 | 62 | 38 |
| | | | | | | | | (60) | (32) |
| 3 | Pd(OAc)₂ | NBS (8.0) | AcOH | 100 | 0 | 0 | 0 | 40 | 60 |
| | | | | | | | | (35) | (44) |
| 4^{[c]} | Pd(OAc)₂ | NBS (8.0) | AcOH | 100 | 0 | 0 | 0 | 44 | 56 |
| 5^{[d]} | Pd(OAc)₂ | NBS (8.0) | AcOH | 100 | 0 | 0 | 0 | 59 | 41 |
| 6 | Pd(OAc)₂ | NBS (8.0) | PivOH | 52 | 45 | 7 | 0 | 0 | 0 |
| | | | | | (36) | | | | |
| 7^{[e]} | Pd(OAc)₂ | NBS (8.0) | AcOH | 100 | 0 | 0 | 0 | 10 | 90 |
| | | | | | | | | (6) | (79) |
| 8^{[f]} | Pd(OAc)₂ | NBS (8.0) | AcOH | 100 | 0 | 0 | 0 | 0 | 99 |
| | | | | | | | | | (89) |

^{[a]}Conditions: 3,6-diphenyl-1,2,4,5-tetrazine (1) (1 equiv), [Pd] (10 mol %), NBS (3-8 equiv), solvent (0.125 M), 110° C., microwave 200 W, under air, 10 min. ¹H NMR yield and isolated yield under bracket.
^{[b]}TFA = trifluoroacetic acid (30% mol.).
^{[c]}PivOH (30% mol).
^{[d]}30 min instead of 10 min.
^{[f]}45 min instead of 10 min.

### A.5 Tetra-Iodination of 3,6-diphenyl-1,2,4,5-tetrazine (1) Under Conditions [a] to [e]

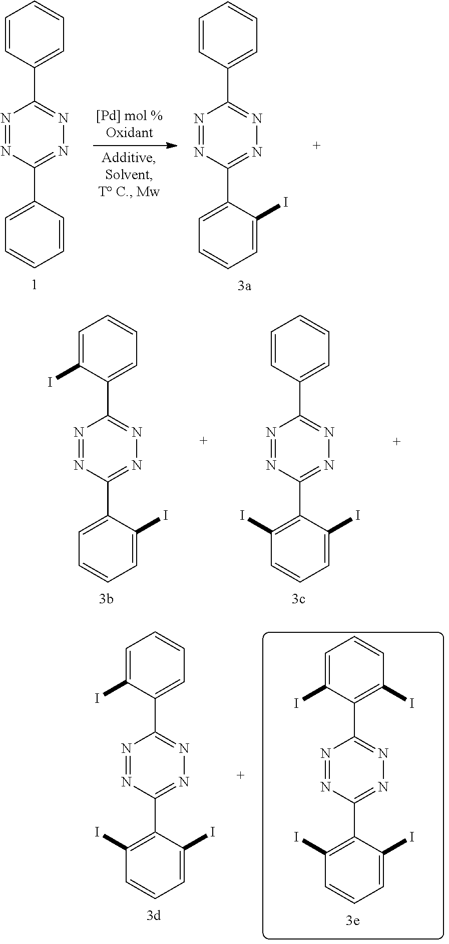

| | | Oxidant | | Conv. | 3a | 3b | 3c | 3d | 3e |
|---|---|---|---|---|---|---|---|---|---|
| Entry | [Pd] | (equiv) | Solvent | (%) | (%) | (%) | (%) | (%) | (%) |
| 1^{[b]} | Pd(OAc)₂ | NIS (2.0) | AcOH | 100 | 13 | 38 | 22 | 27 | 0 |
| | | | | | | (17) | (13) | | |
| 2 | Pd(OAc)₂ | NIS (8.0) | AcOH | 100 | 0 | 0 | 0 | 67 | 33 |
| | | | | | | | | (7) | (31) |
| 3 | Pd(OAc)₂ | NIS (10.0) | AcOH | 100 | 0 | 0 | 0 | 52 | 38^{[c]} |
| 4 | Pd(OAc)₂ | NIS (12.0) | AcOH | 100 | 0 | 0 | 0 | 0 | 26^{[c]} |
| 5^{[d]} | Pd(OAc)₂ | NIS (12.0) | CH₃NO₂ | 100 | 0 | 0 | 0 | 58 | 42 |
| | | | | | | | | (9) | (42) |
| 6^{[d, e]} | Pd(OAc)₂ | NIS (12.0) | CH₃NO₂ | 100 | 0 | 0 | 0 | 78 | 22 |
| | | | | | | | | (15) | (20) |

^{[a]}Conditions: 3,6-diphenyl-1,2,4,5-tetrazine (1) (1 equiv), [Pd] (10 mol %), NIS (8-12 equiv), solvent (0.125 M), 110° C., microwave 200 W, under air, 10 min. ¹H NMR yield and isolated yield under bracket.
^{[b]}120° C. instead of 110° C.
^{[c]}3-(2-acetoxy-6-iodophenyl)-6-(2,6-diiodophenyl)-1,2,4,5-tetrazine was detected by ¹H NMR et GC-MS.
^{[d]}100° C. instead of 110° C.
^{[e]}PivOH (30% mol).

### A.6 Tetra-Chlorination of 3,6-diphenyl-1,2,4,5-tetrazine (1) Under Conditions [a] to [e]

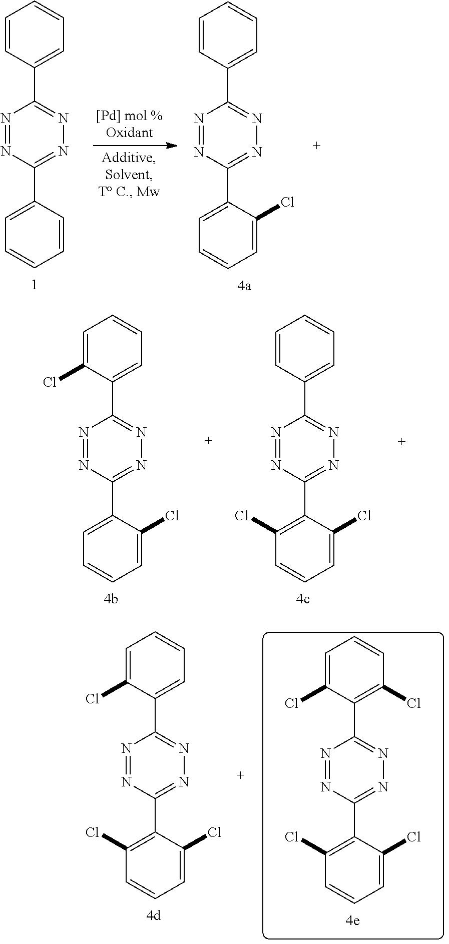

| | | Oxidant | | Conv. | 4a | 4b | 4c | 4d | 4e |
|---|---|---|---|---|---|---|---|---|---|
| Entry | [Pd] | (equiv) | Solvent | (%) | (%) | (%) | (%) | (%) | (%) |
| 1^{[b]} | Pd(OAc)₂ | NIS (2.0) | AcOH | 100 | 13 | 38 | 22 | 27 | 0 |
| | | | | | | (17) | (13) | | |
| 2 | Pd(OAc)₂ | NIS (8.0) | AcOH | 100 | 0 | 0 | 0 | 67 | 33 |
| | | | | | | | | (7) | (31) |
| 3 | Pd(OAc)₂ | NIS (10.0) | AcOH | 100 | 0 | 0 | 0 | 52 | 38^{[c]} |
| 4 | Pd(OAc)₂ | NIS (12.0) | AcOH | 100 | 0 | 0 | 0 | 0 | 26^{[c]} |
| 5^{[d]} | Pd(OAc)₂ | NIS (12.0) | CH₃NO₂ | 100 | 0 | 0 | 0 | 58 | 42 |
| | | | | | | | | (9) | (42) |
| 6^{[d, e]} | Pd(OAc)₂ | NIS (12.0) | CH₃NO₂ | 100 | 0 | 0 | 0 | 78 | 22 |
| | | | | | | | | (15) | (20) |

^{[a]}Conditions: 3,6-diphenyl-1,2,4,5-tetrazine (1) (1 equiv), [Pd] (10 mol %), NCS (4-12 equiv), solvent (0.125 M), 110° C., microwave 200 W, under air, 45 min. ¹H NMR yield and isolated yield under bracket.
^{[c]}10 min instead of 45 min.
^{[d]}120° C. instead of 110° C.
^{[e]}PivOH (30% mol).

### A.7 Di- and Tri-Halogenation of 3,6-diphenyl-1,2,4,5-tetrazine (1)

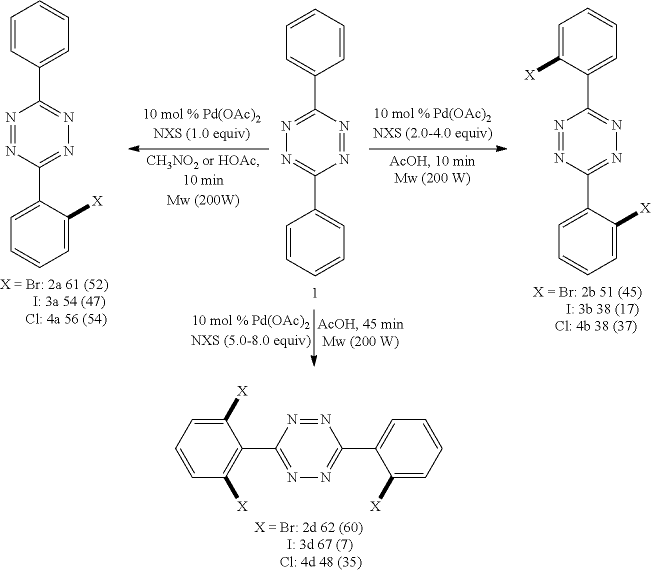

This is a fast synthetic access to halogenated s-aryltetrazines towards a wider set of polyhalogenated compounds including the symmetrical ortho-dihalogenated (2b-4b) and the dissymmetrical ortho-trihalogenated (2d-4d). These latter being already pertinent candidates for further construction of dissymmetrized s-tetrazines. The dibrominated aryltetrazine (2b), which is a useful precursor for the synthesis of benzo[a]acecorannulene bowl-shaped fullerene materials, was obtained using 2 equiv of NBS with [Pd(OAc)₂] as the catalyst in AcOH in 10 min at 110° C. with 45% isolated yield. Its iodinated analogue (3b) was synthesized under similar conditions using a slightly higher temperature of 120° C. Side products 3-(2,6-diiodophenyl)-6-phenyl-1,2,4,5-tetrazine (3c) (22%) and (3d) (27%) makes workup more difficult. The dichlorinated s-aryltetrazine (4b) was synthesized again in only a few minutes using 4 equiv of NCS at 120° C., and was isolated in 37% yield.

One-step tri halogenation of (1) was efficiently achieved from larger amounts of electrophilic halide sources and longer reaction periods under microwave heating (still lesser than one hour reaction time). Tris-brominated s-aryltetrazine 3-(2,6-dibromophenyl)-6-(2-bromophenyl)-1,2,4,5-tetrazine (2d) was isolated in 60% yield under pure form. The access to tris-iodinated s-aryltetrazine (3d) was found troublesome since from a satisfactory 67% conversion of (1) pure (3d) was isolated in only low 7% yield due to its sensitiveness to column chromatography. Tris-chlorinated (4d) was isolated in pure form in 35% yield from a 48% yield conversion.

### B—General Procedure of Functionalization of Tetrazine

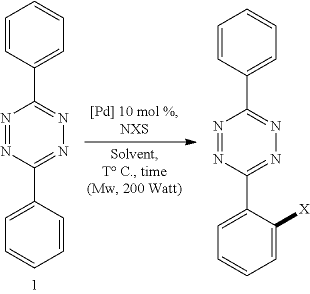

As a typical experiment, the tetrazine (1.0 eq., 0.25 mmol), halogenated source (X eq.), and palladium source (10 mol %) were introduced in a 10 mL microwave reaction vessel, equipped with a magnetic stirring bar. The solvent (mL, 0.125 M) was added, and the reaction mixture was heated in the microwave at T° C. for corresponding reaction time (200 W, 2 min ramp). After cooling down to room temperature, the reaction mixture was diluted with dichloromethane, and was washed three times with water+3% of TEA (or Na₂S₂O₃ when NIS was involved). The combined organic layer was washed with water and dried over MgSO₄. The solvent was removed in vacuo and the residue was analysed by NMR to determine the conversion of the halogenated product. Then, the crude product was purified by silica gel column chromatography using an appropriate ratio of the eluent. For elemental analysis, the product was recrystallized with a slow diffusion of dichloromethane into heptane (RPE quality).

According to the above-described process, the following compounds are obtained:
3-(2-bromophenyl)-6-phenyl-1,2,4,5-tetrazine (2a),
3,6-bis(2-bromophenyl)-1,2,4,5-tetrazine (2b),
3-(2,6-dibromophenyl)-6-(2-bromophenyl)-1,2,4,5-tetrazine (2d),
3,6-bis(2,6-dibromophenyl)-1,2,4,5-tetrazine (2e),
3-(2-iodophenyl)-6-phenyl-1,2,4,5-tetrazine (3a),
3,6-bis(2-iodophenyl)-1,2,4,5-tetrazine (3b),
3-(2,6-diiodophenyl)-6-(2-iodophenyl)-1,2,4,5-tetrazine (3d),
3,6-bis(2,6-diiodophenyl)-1,2,4,5-tetrazine (3e),
3-(2-chlorophenyl)-6-phenyl-1,2,4,5-tetrazine (4a),
3,6-bis(2-chlorophenyl)-1,2,4,5-tetrazine (4b),
3-(2,6-dichlorophenyl)-6-(2-chlorophenyl)-1,2,4,5-tetrazine (4d), and
3,6-bis(2,6-dichlorophenyl)-1,2,4,5-tetrazine (4e).

### C—Synthesis of Unequally Polyhalogenated Compounds of Formula (Ia)

From the difluorinated s-aryltetrazine (6b), monobromination and monoiodination proceeded selectively in only ten minutes with precisely 2 equiv of N-halosuccinimide. Products (12a) and (12b) were then isolated pure in 33% and 54% yield, respectively. For the more demanding selective monochlorination of (6b) the reaction time was not extended but instead 4 equiv of NCS was used to isolate (12c) in 50% yield. Monobromination and monoiodination of the dichlorinated s-aryltetrazine (4b) were found easier and the trihalogenated products (13a) and (13b) were converted in 71% and 75%. A more challenging two-steps access to tri halogenated species is the reverse process where di halogenation follows mono halogenation of s-aryltetrazine. This was done for dibromination and dichlorination of (6a) to reach targets (14a) and (14b).

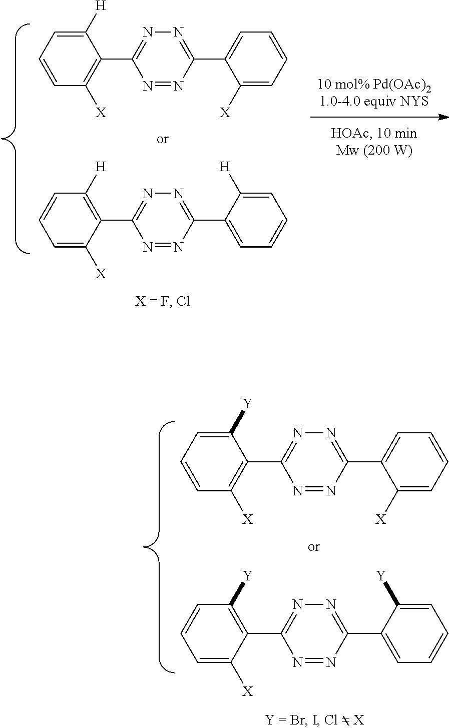

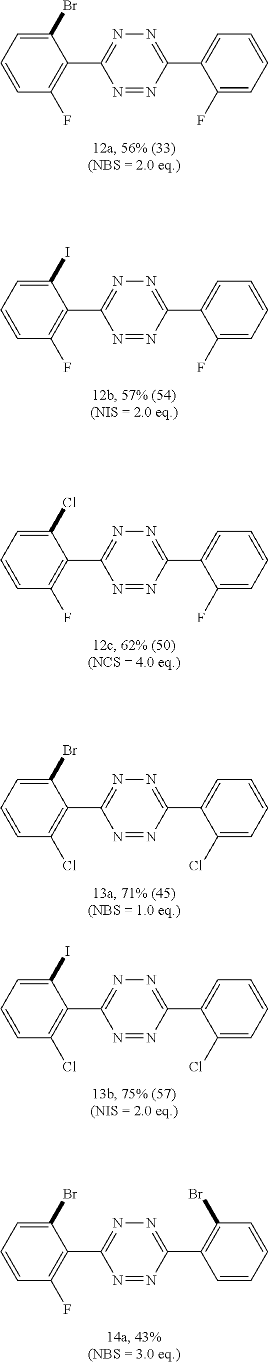

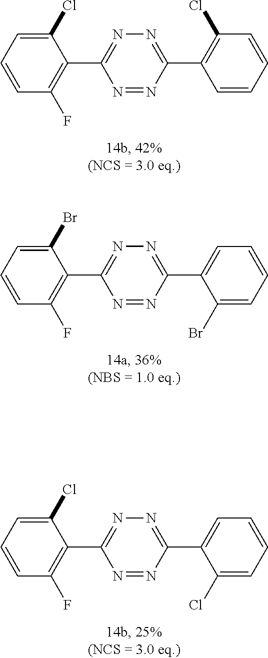

[a] Conditions: 3,6-diaryl-1,2,4,5-tetrazine derivative (1 equiv), Pd(OAc)₂ (10 mol %), [NYS]: NBS or NIS or NCS (2.0 to 4.0 equiv), solvent (0.125 M), 120° C., microwave 200 W, under air, 10 min. ¹H NMR yield and isolated yield under bracket.

Combining the above-described one-step mono-, di-, and tri halogenation reactions, successively, allows forming the polyhalogenated compounds described below, that can be unequally functionalized with up to three different functions in the four positions.

### 3-(2-bromo-6-fluorophenyl)-6-(2,6-dibromophenyl)-1,2,4,5-tetrazine (15)

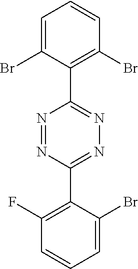 [SMILES: Fc1cccc(Br)c1-c1nnc(-c2c(Br)cccc2Br)nn1]

Rf=0.59 (Dichloromethane-Heptane=1:1 (v/v)).

¹H NMR (300 MHz, CDCl₃): δ (ppm)=7.78 (d, J=8.10 Hz, 2H), 7.63 (dt, J=8.10, 0.92 Hz, 1H), 7.54-7.47 (m, 1H), 7.39-7.30 (m, 2H).

¹⁹F NMR (282 MHz, CDCl₃): δ (ppm)=−109.9.

¹³C NMR (75 MHz, CDCl₃): δ (ppm)=167.4, 163.9 (d, J=1.3 Hz), 162.6 (d, J=256.3 Hz), 135.6, 133.4 (d, J=9.0 Hz), 133.0, 132.0, 129.0 (d, J=3.6 Hz), 123.7 (d, J=17.8 Hz), 123.6, 123.5 (d, J=2.6 Hz), 115.6 (d, J=21.3 Hz).

Elemental analysis: Calcd (%) for C₁₄H₆Br₃FN₄: C, 34.39, H, 1.24, N, 11.46. Found: C, 34.62, H, 1.47, N, 11.15.

HRMS+p ESI (m/z) [M+H⁺] Calcd for C₁₄H₆Br₃FN₄: 486.820. Found: m/z=486.821.

### 3-(2-iodo-6-fluorophenyl)-6-(2,6-diiodophenyl)-1,2,4,5-tetrazine (16)

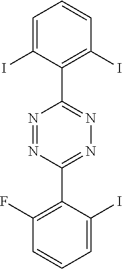 [SMILES: Fc1cccc(I)c1-c1nnc(-c2c(I)cccc2I)nn1]

Rf=047 (Dichloromethane-Heptane=1:1 (v/v)).

¹H NMR (300 MHz, CDCl₃): δ (ppm)=8.05 (d, J=8.00 Hz, 2H), 7.91-7.85 (m, 1H), 7.37-7.33 (m, 2H), 6.99 (t, J=8.00 Hz, 2H).

¹⁹F NMR (282 MHz, CDCl₃): δ (ppm)=−108.6.

¹³C NMR (75 MHz, CDCl₃): δ (ppm)=170.7, 165.3 (d, J=1.4 Hz), 162.0 (d, J=256.9 Hz), 142.3, 139.1, 135.4 (d, J=3.6 Hz), 133.8 (d, J=9.0 Hz), 133.2, 127.11 (d, J=17.3 Hz), 116.4 (d, J=21.3 Hz), 97.1 (d, J=0.9 Hz), 96.0.

Elemental analysis: Calcd (%) for C₁₄H₆FI₃N₄: C, 26.69, H, 0.96, N, 8.89. Found: C, 26.86, H, 1.09, N, 8.43.

HRMS+p ESI (m/z) [M+Na⁺] Calcd for C₁₄H₆FI₃N₄: 652.760. Found: m/z=652.760.

### 3-(2-bromo-6-fluorophenyl)-6-(2,6-dichlorophenyl)-1,2,4,5-tetrazine (17)

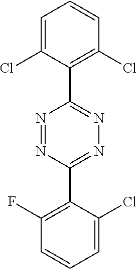 [SMILES: Fc1cccc(Cl)c1-c1nnc(-c2c(Cl)cccc2Cl)nn1]

Rf=0.57 (Dichloromethane-Heptane=1:1 (v/v)).

¹H NMR (300 MHz, CDCl₃): δ (ppm)=7.54-7.42 (m, 4H), 7.39 (td, J=8.10, 0.92 Hz, 1H), 7.22 (dt, J=8.10, 0.92 Hz, 1H).

¹⁹F NMR (282 MHz, CDCl₃): δ (ppm)=−111.0.

¹³C NMR (75 MHz, CDCl₃): δ (ppm)=165.3, 163.2 (d, J=1.4 Hz), 162.8 (d, J=256.9 Hz), 135.1, 134.8 (d, J=3.6 Hz), 133.1 (d, J=9.0 Hz), 132.4, 132.0, 128.4, 126.0 (d, J=3.6 Hz), 121.8 (d, J=17.3 Hz), 115.0 (d, J=21.3 Hz).

Elemental analysis: Calcd (%) for C₁₄H₆Cl₃FN₄: C, 47.29, H, 1.70, N, 15.76. Found: C, 47.58, H, 1.32, N, 15.45.

HRMS+p ESI (m/z) [M+H⁺] Calcd for C₁₄H₆Cl₃FN₄: 354.971. Found: m/z=354.970.

### 3-(2-bromo-6-chlorophenyl)-6-(2,6-dibromophenyl)-1,2,4,5-tetrazine (18)

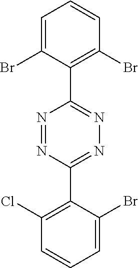 [SMILES: Clc1cccc(Br)c1-c1nnc(-c2c(Br)cccc2Br)nn1]

Rf=0.68 (Dichloromethane-Heptane=2:1 (v/v)).

¹H NMR (300 MHz, CDCl₃): δ (ppm)=7.78 (d, J=8.10 Hz, 2H), 7.73 (dd, J=8.10, 0.90 Hz, 1H), 7.61 (dd, J=8.10, 0.90 Hz, 1H), 7.44 (t, J=8.10 Hz, 1H), 7.39 (t, J=8.10 Hz, 1H).

¹³C NMR (75 MHz, CDCl₃): δ (ppm)=167.4, 166.3, 135.6, 134.8, 133.9, 132.9, 132.7, 132.0, 131.5, 128.9, 123.7, 123.5.

Elemental analysis: Calcd (%) for C₁₄H₆Br₃ClN₄: C, 33.27, H, 1.20, N, 11.09. Found: C, 33.78, H, 1.54, N, 10.69.

HRMS+p ESI (m/z) [M+H⁺] Calcd for C₁₄H₆Br₃ClN₄: 502.790. Found: m/z=502.792.

### 3,6-bis(2-bromo-6-fluorophenyl)-1,2,4,5-tetrazine (19)

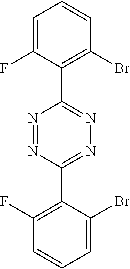 [SMILES: Fc1cccc(Br)c1-c1nnc(-c2c(F)cccc2Br)nn1]

Rf=0.61 (dichloromethane-heptane=7:3 (v/v)).

¹H NMR (300 MHz, CDCl₃): δ (ppm)=7.62 (dt, J=8.10, 0.92 Hz, 2H), 7.51 (t, J=8.31 Hz, 2H), 7.48 (t, J=8.33 Hz, 2H), 7.35-7.29 (m, 2H).

¹⁹F NMR (282 MHz, CDCl₃): δ (ppm)=−109.9.

¹³C NMR (75 MHz, CDCl₃): δ (ppm)=164.2 (d, J=1.3 Hz), 162.9 (d, J=256.3 Hz), 133.6 (d, J=9.1 Hz), 129.3 (d, J=3.6 Hz), 123.8 (d, J=17.3 Hz), 123.7 (d, J=2.6 Hz), 115.8 (d, J=21.3 Hz).

Elemental analysis: Calcd (%) for C₁₄H₆Br₂F₂N₄: C, 39.29, H, 1.41, N, 13.09. Found: C, 39.24, H, 1.75, N, 11.95.

HRMS+p ESI (m/z) [M+H⁺] Calcd for C₁₄H₆Br₂F₂N₄: 426.900. Found: m/z=426.900.

### 3,6-bis(2-iodo-6-fluorophenyl)-1,2,4,5-tetrazine (20)

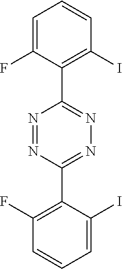 [SMILES: Fc1cccc(I)c1-c1nnc(-c2c(F)cccc2I)nn1]

Rf=0.58 (dichloromethane-heptane=7:3 (v/v)).

¹H NMR (300 MHz, CDCl₃): δ (ppm)=7.90-7.84 (m, 2H), 7.38-7.32 (m, 4H).

¹⁹F NMR (282 MHz, CDCl₃): δ (ppm)=−108.6.

¹³C NMR (75 MHz, CDCl₃): δ (ppm)=165.5 (d, J=1.4 Hz), 162.2 (d, J=256.9 Hz), 135.6 (d, J=3.7 Hz), 133.9 (d, J=8.7 Hz), 127.2 (d, J=16.6 Hz), 116.6 (d, J=21.3 Hz), 97.2 (d, J=0.9 Hz).

Elemental analysis: Calcd (%) for C₁₄H₆I₂F₂N₄: C, 32.21, H, 1.16, N, 10.73. Found: C, 33.79, H, 1.58, N, 10.38.

HRMS+p ESI (m/z) [M+H⁺] Calcd for C₁₄H₆F₂I₂N₄: 522.872. Found: m/z=522.871.

### 3,6-bis(2-chloro-6-fluorophenyl)-1,2,4,5-tetrazine (21)

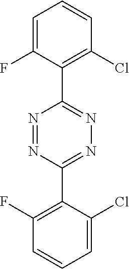 [SMILES: Fc1cccc(Cl)c1-c1nnc(-c2c(F)cccc2Cl)nn1]

Rf=0.59 (dichloromethane-heptane=7:3 (v/v)).

¹H NMR (300 MHz, CDCl₃): δ (ppm)=7.58 (t, J=8.27 Hz, 1H), 7.56 (t, J=8.23 Hz, 1H), 7.45 (dt, J=8.17, 1.03 Hz, 2H), 7.31-7.25 (m, 2H).

¹⁹F NMR (282 MHz, CDCl₃): δ (ppm)=−111.0.

¹³C NMR (75 MHz, CDCl₃): δ (ppm)=163.3 (d, J=1.3 Hz), 163.0 (d, J=255.7 Hz), 135.1 (d, J=3.5 Hz), 133.3 (d, J=9.5 Hz), 126.2 (d, J=3.6 Hz), 121.9 (d, J=17.3 Hz), 115.2 (d, J=21.3 Hz).

Elemental analysis: Calcd (%) for C₁₄H₆Cl₂F₂N₄: C, 49.58, H, 1.78, N, 16.52. Found: C, 46.64, H, 2.08, N, 14.68.

HRMS+p ESI (m/z) [M+Na⁺] Calcd for C₁₄H₆Cl₂F₂N₄: 360.982. Found: m/z=360.982.

### 3,6-bis(2-bromo-6-chlorophenyl)-1,2,4,5-tetrazine (22)

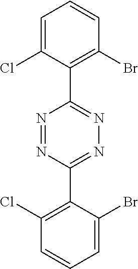 [SMILES: Clc1cccc(Br)c1-c1nnc(-c2c(Cl)cccc2Br)nn1]

Rf=0.49 (dichloromethane-heptane=1:1 (v/v)).

¹H NMR (300 MHz, CDCl₃): δ (ppm)=7.75 (dd, J=8.08, 1.03 Hz, 2H), 7.60 (dd, J=8.16, 1.03 Hz, 2H), 7.44 (t, J=8.11 Hz, 2H).

¹³C NMR (75 MHz, CDCl₃): δ (ppm)=166.5, 135.1, 134.1, 132.8, 131.6, 129.1, 123.9.

Elemental analysis: Calcd (%) for C₁₄H₆Br₂Cl₂N₄: C, 36.48, H, 1.31, N, 12.16. Found: C, 35.61, H, 1.71, N, 10.90.

HRMS+p ESI (m/z) [M+H⁺] Calcd for C₁₄H₆Br₂Cl₂N₄: 458.840. Found: m/z=458.840.

### 3,6-bis(2-chloro-6-iodophenyl)-1,2,4,5-tetrazine (23)

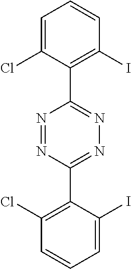 [SMILES: Clc1cccc(I)c1-c1nnc(-c2c(Cl)cccc2I)nn1]

Rf=0.37 (dichloromethane-heptane=1:1 (v/v)).

¹H NMR (300 MHz, CDCl₃): δ (ppm)=7.77 (dd, J=8.00, 0.98 Hz, 2H), 7.64 (dd, J=8.14, 0.97 Hz, 2H), 7.27 (t, J=8.06 Hz, 2H). ¹³C NMR (75 MHz, CDCl₃): δ (ppm)=168.2, 137.9, 137.6, 134.1, 133.0, 129.9, 97.4.

Elemental analysis: Calcd (%) for C₁₄H₆Cl₂I₂N₄: C, 30.30, H, 1.09, N, 10.10. Found: C, 30.09, H, 1.06, N, 10.05.

HRMS+p ESI (m/z) [M+H⁺] Calcd for C₁₄H₆Cl₂I₂N₄: 554.813. Found: m/z=554.812.

### 3-(2-bromophenyl)-6-(2-fluorophenyl)-1,2,4,5-tetrazine (24)

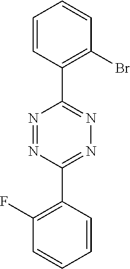 [SMILES: Fc1ccccc1-c1nnc(-c2ccccc2Br)nn1]

Rf=0.33 (Dichloromethane-Heptane=1:1 (v/v)).

¹H NMR (300 MHz, CDCl₃): δ (ppm)=8.40 (td, J=7.9, 1.8 Hz, 1H), 8.05 (dd, J=7.7, 1.7 Hz, 1H), 7.83 (dd, J=7.9, 1.8 Hz, 1H), 7.69-7.61 (m, 1H), 7.58 (td, J=7.6, 1.2 Hz, 1H), 7.49 (dd, J=7.9, 1.8 Hz, 1H), 7.43 (dd, J=8.0, 1.1 Hz, 1H), 7.40-7.32 (m, 1H).

¹⁹F NMR (282 MHz, CDCl₃): δ (ppm)=−111.5.

¹³C NMR (75 MHz, CDCl₃): δ (ppm)=165.7, 163.4 (d, J=260.0 Hz), 163.1 (d, J=5.8 Hz), 134.4, 134.4 (d, J=8.8 Hz), 133.5, 132.6, 132.3, 131.6 (d, J=0.9 Hz), 127.9, 124.9 (d, J=3.9 Hz), 122.4, 120.4 (d, J=9.8 Hz), 117.6 (d, J=21.6 Hz).

Elemental analysis: Calcd (%) for C₁₄H₈BrFN₄: C, 50.78, H, 2.44, N, 16.92. Found: C, 50.56, H, 3.04, N, 16.51.

HRMS+p ESI (m/z) [M+Na⁺] Calcd for C₁₄H₈BrFN₄: 352.980. Found: m/z=352.980.

### 3-(2-bromo-6-fluorophenyl)-6-phenyl-1,2,4,5-tetrazine (25)

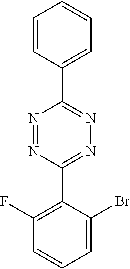 [SMILES: Fc1cccc(Br)c1-c1nnc(-c2ccccc2)nn1]

Rf=0.46 (Dichloromethane-Heptane=1:1 (v/v)).

¹H NMR (300 MHz, CDCl₃): δ (ppm)=8.75-8.72 (m, 2H), 7.69-7.60 (m, 4H), 7.51-7.44 (m, 1H), 7.30 (td, J=8.10, 0.90 Hz).

¹⁹F NMR (282 MHz, CDCl₃): δ (ppm)=−110.1.

¹³C NMR (75 MHz, CDCl₃): δ (ppm)=164.3 (d, J=1.5 Hz), 163.9, 163.0 (d, J=256.0 Hz), 133.4, 133.3 (d, J=9.1 Hz), 131.5, 129.6, 129.2 (d, J=3.6 Hz), 128.7, 124.1 (d, J=18.0 Hz), 123.8 (d, J=2.6 Hz), 115.7 (d, J=21.5 Hz).

Elemental analysis: Calcd (%) for C₁₄H₈BrFN₄: C, 50.78, H, 2.44, N, 16.92.

Found: C, 50.56, H, 3.04, N, 16.51.

HRMS+p ESI (m/z) [M+Na⁺] Calcd for C₁₄H₈BrFN₄: 352.980. Found: m/z=352.980.

### 3-(2-fluorophenyl)-6-(2-iodophenyl)-1,2,4,5-tetrazine (26)

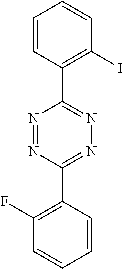 [SMILES: Fc1ccccc1-c1nnc(-c2ccccc2I)nn1]

Rf=0.36 (Dichloromethane-Heptane=1:1 (v/v)).

¹H NMR (300 MHz, CDCl₃): δ (ppm)=8.41 (td, J=1.80, 7.63.Hz, 1H), 8.13 (dd, J=1.01, 8.00 Hz, 1H), 8.03 (dd, J=1.63, 7.75 Hz, 1H), 7.69-7.58 (m, 2H), 7.42 (td, J=1.13, 7.72 Hz, 1H), 7.36-7.27 (m, 2H).

⁹F NMR (282 MHz, CDCl₃): δ (ppm)=−111.5.

¹³C NMR (75 MHz, CDCl₃): δ (ppm)=166.4, 163.4 (d, J=260.1 Hz), 163.2 (d, J=5.9 Hz), 141.2, 136.8, 134.4 (d, J=8.8 Hz), 132.4, 131.7, 131.6 (d, J=1.0 Hz), 128.7, 124.9 (d, J=3.9 Hz), 120.5 (d, J=9.8 Hz), 117.6 (d, J=21.6 Hz), 95.6.

Elemental analysis: Calcd (%) for C₁₄H₈FIN₄: C, 44.47, H, 2.13, N, 14.82. Found: C, 44.74, H, 2.84, N, 14.23.

HRMS+p ESI (m/z) [M+H⁺] Calcd for C₁₄H₈FIN₄: 378.985. Found: m/z=378.985.

### 3-(2-fluoro-6-iodophenyl)-6-phenyl-1,2,4,5-tetrazine (27)

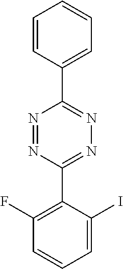 [SMILES: Fc1cccc(I)c1-c1nnc(-c2ccccc2)nn1]

Rf=0.42 (Dichloromethane-Heptane=1:1 (v/v)).

¹H NMR (300 MHz, CDCl₃): δ (ppm)=8.76-8.72 (m, 2H), 7.90-7.84 (m, 1H), 7.70-7.61 (m, 3H), 7.34-7.30 (m, 2H).

⁹F NMR (282 MHz, CDCl₃): δ (ppm)=−108.9.

### 3-(2-chlorophenyl)-6-(2-fluorophenyl)-1,2,4,5-tetrazine (28)

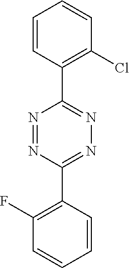 [SMILES: Fc1ccccc1-c1nnc(-c2ccccc2Cl)nn1]

Rf=0.35 (Dichloromethane-Heptane=1:1 (v/v)).

¹H NMR (300 MHz, CDCl₃): δ (ppm)=8.40 (td, J=7.65, 1.80 Hz, 1H), 8.10 (td, J=7.00, 1.80 Hz, 1H), 7.69-7.50 (m, 4H), 7.43 (td, J=7.65, 1.80 Hz, 1H), 7.36 (ddd, J=10.8, 7.65, 1.10 Hz, 1H).

¹⁹F NMR (282 MHz, CDCl₃): δ (ppm)=−111.5.

¹³C NMR (75 MHz, CDCl₃): δ (ppm)=165.0, 163.4 (d, J=260.0 Hz), 163.1 (d, J=5.8 Hz), 134.4 (d, J=9.9 Hz), 133.8, 132.6, 132.2, 131.6 (d, J=0.9 Hz), 131.2, 127.4, 124.9 (d, J=3.9 Hz), 120.5 (d, J=9.8 Hz), 117.6 (d, J=21.6 Hz).

Elemental analysis: Calcd (%) for C₁₄H₈ClFN₄: C, 58.65, H, 2.81, N, 19.54. Found: C, 58.41, H, 2.68, N, 19.32.

HRMS+p ESI (m/z) [M+Na⁺] Calcd for C₁₄H₈ClFN₄: 309.031. Found: m/z=309.031.

### 3-(2-chloro-6-fluorophenyl)-6-phenyl-1,2,4,5-tetrazine (29)

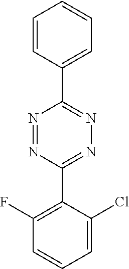 [SMILES: Fc1cccc(Cl)c1-c1nnc(-c2ccccc2)nn1]

Rf=0.48 (Dichloromethane-Heptane=1:1 (v/v)).

¹H NMR (300 MHz, CDCl₃): δ (ppm)=8.74-8.71 (m, 2H), 7.69-7.61 (m, 3H), 7.58-7.50 (m, 1H), 7.44 (td, J=7.00, 1.80 Hz, 1H), 7.26 (td, J=7.00, 1.80 Hz, 1H).

¹⁹F NMR (282 MHz, CDCl₃): δ (ppm)=−111.2.

¹³C NMR (75 MHz, CDCl₃): δ (ppm)=163.9, 163.3 (d, J=1.5 Hz), 163.0 (d, J=255.0 Hz), 135.1 (d, J=3.5 Hz), 133.4, 132.9 (d, J=9.5 Hz), 131.5, 129.5, 128.7, 126.2 (d, J=3.6 Hz), 122.2 (d, J=17.4 Hz), 115.7 (d, J=21.5 Hz).

Elemental analysis: Calcd (%) for C₁₄H₈ClFN₄: C, 58.65, H, 2.81, N, 19.54. Found: C, 58.41, H, 2.68, N, 19.32.

HRMS+p ESI (m/z) [M+Na⁺] Calcd for C₁₄H₈ClFN₄: 309.031. Found: m/z=309.031.

### 3-(2-bromophenyl)-6-(2-chlorophenyl)-1,2,4,5-tetrazine (30)

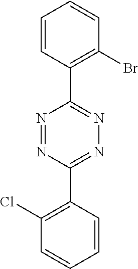 [SMILES: Clc1ccccc1-c1nnc(-c2ccccc2Br)nn1]

Rf=0.33 (Dichloromethane-Heptane=2:3 (v/v)).

¹H NMR (300 MHz, CDCl₃): δ (ppm)=8.13-8.05 (m, 2H), 7.84 (dd, J=7.00, 1.80 Hz, 1H), 7.67-7.46 (m, 5H).

¹³C NMR (75 MHz, CDCl₃): δ (ppm)=165.6, 164.9, 134.4, 133.9, 133.4, 132.7, 132.6, 132.3, 132.3, 131.5, 131.2, 127.9, 127.4, 122.5.

Elemental analysis: Calcd (%) for C₁₄H₈BrClN₄: C, 48.38, H, 2.32, N, 16.12. Found: C, 48.59, H, 2.53, N, 15.88.

HRMS+p ESI (m/z) [M+H⁺] Calcd for C₁₄H₈BrClN₄: 346.969. Found: m/z=346.970.

### 3-(2-bromo-6-chlorophenyl)-6-phenyl-1,2,4,5-tetrazine (31)

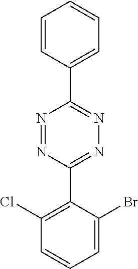 [SMILES: Clc1cccc(Br)c1-c1nnc(-c2ccccc2)nn1]

Rf=0.28 (Dichloromethane-Heptane=2:3 (v/v)).

¹H NMR (300 MHz, CDCl₃): δ (ppm)=8.76-8.73 (m, 2H), 7.71 (dd, J=8.10, 1.00 Hz, 1H), 7.70-7.62 (m, 3H), 7.59 (dd, J=8.10, 1.00 Hz, 1H), 7.41 (t, J=8.12 Hz, 1H).

### 3-(2-chlorophenyl)-6-(2-iodophenyl)-1,2,4,5-tetrazine (32)

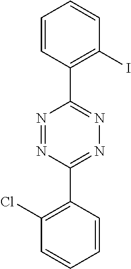 [SMILES: Clc1ccccc1-c1nnc(-c2ccccc2I)nn1]

Rf=0.36 (Dichloromethane-Heptane=1:1 (v/v)).

¹H NMR (300 MHz, CDCl₃): δ (ppm)=8.15-8.11 (m, 2H), 8.06 (dd, J=7.75, 1.60 Hz, 1H), 7.67-7.51 (m, 4H), 7.30 (td, J=7.75, 1.60 Hz, 1H).

¹³C NMR (75 MHz, CDCl₃): δ (ppm)=166.3, 164.9, 141.2, 136.7, 133.9, 132.7, 132.5, 132.3, 131.7, 131.5, 131.2, 128.7, 127.36, 95.7.

Elemental analysis: Calcd (%) for C₁₄H₈ClIN₄: C, 42.61, H, 2.04, N, 14.20. Found: C, 42.98, H, 2.57, N, 11.86.

HRMS+p ESI (m/z) [M+H⁺] Calcd for C₁₄H₈ClIN₄: 394.955. Found: m/z=394.955.

### 3-(2-chloro-6-iodophenyl)-6-phenyl-1,2,4,5-tetrazine (33)

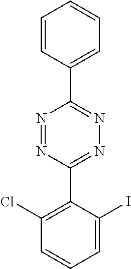 [SMILES: Clc1cccc(I)c1-c1nnc(-c2ccccc2)nn1]

Rf=0.47 (Dichloromethane-Heptane=1:1 (v/v)).

¹H NMR (300 MHz, CDCl₃): δ (ppm)=8.77-8.74 (m, 2H); 7.96 (dd, J=8.00, 1.00 Hz, 1H), 7.70-7.63 (m, 3H), 7.61 (dd, J=8.00, 1.00 Hz, 1H), 7.24 (t, J=8.00 Hz, 1H).

### 3-(2-bromophenyl)-6-(2-iodophenyl)-1,2,4,5-tetrazine (34)

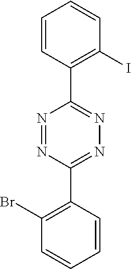 [SMILES: Brc1ccccc1-c1nnc(-c2ccccc2I)nn1]

Rf=0.36 (Dichloromethane-Heptane=1:1 (v/v)).

¹H NMR (300 MHz, CDCl₃): δ (ppm)=8.13 (dd, J=7.98, 1.04 Hz, 1H), 8.10-8.05 (m, 2H), 7.84 (dd, J=7.95, 1.16 Hz, 1H), 7.65-7.56 (m, 2H), 7.49 (td, J=7.54, 1.77 Hz, 1H) 7.30 (dd, J=7.58, 1.70 Hz, 1H).

¹³C NMR (75 MHz, CDCl₃): δ (ppm)=166.3, 165.5, 141.0, 136.7, 134.4, 133.4, 123.6, 132.5, 132.3, 131.6, 128.7, 127.9, 122.5, 95.7.

Elemental analysis: Calcd (%) for C₁₄H₈BrIN₄: C, 38.30, H, 1.84, N, 12.76. Found: C, 38.86, H, 2.42, N, 12.14.

HRMS+p ESI (m/z) [M+Na⁺] Calcd for C₁₄H₈BrIN₄: 460.887. Found: m/z=460.887.

### 3-(2-bromo-6-iodophenyl)-6-phenyl-1,2,4,5-tetrazine (35)

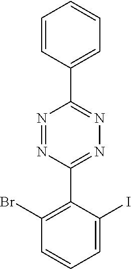 [SMILES: Brc1cccc(I)c1-c1nnc(-c2ccccc2)nn1]

Rf=0.47 (Dichloromethane-Heptane=1:1 (v/v)).

¹H NMR (300 MHz, CDCl₃): δ (ppm)=8.77-8.74 (m, 2H), 7.01 (dd, J=8.00, 1.00 Hz, 1H), 7.79 (dd, J=8.10, 1.00 Hz, 1H), 7.70-7.62 (m, 3H), 7.16 (t, J=8.04 Hz, 1H).

### 3-(2-bromo-6-fluorophenyl)-6-(2-fluorophenyl)-1,2,4,5-tetrazine (12a)

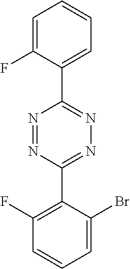 [SMILES: Fc1ccccc1-c1nnc(-c2c(F)cccc2Br)nn1]

Rf=0.52 (dichloromethane-heptane=7:3 (v/v)).

¹H NMR (300 MHz, CDCl₃): δ (ppm)=8.42 (td, J=7.53, 1.69 Hz, 1H), 7.70-7.64 (m, 1H), 7.62 (dt, J=8.14, 0.98 Hz, 1H), 7.52-7.27 (m, 4H).

¹⁹F NMR (282 MHz, CDCl₃): δ (ppm)=−110.1, −111.1.

¹³C NMR (75 MHz, CDCl₃): δ (ppm)=164.0 (d, J=5.9 Hz), 163.5 (d, J=260.6 Hz), 163.5 (d, J=1.3 Hz), 163.0 (d, J=256.0 Hz), 134.6 (d, J=8.8 Hz), 133.3 (d, J=9.2 Hz), 131.8 (d, J=0.8 Hz), 129.1 (d, J=3.6 Hz), 124.9 (d, J=3.9 Hz), 124.0 (d, J=17.2 Hz), 123.8 (d, J=2.6 Hz), 120.4 (d, J=9.7 Hz), 117.7 (d, J=21.6 Hz), 115.6 (d, J=21.4 Hz).

Elemental analysis: Calcd (%) for C₁₄H₇BrF₂N₄: C, 48.16, H, 2.02, N, 16.05. Found: C, 50.53, H, 3.34, N, 13.56.

HRMS+p ESI (m/z) [M+H⁺] Calcd for C₁₄H₇BrF₂N₄: 348.989. Found: m/z=348.988.

### 3-(2-fluoro-6-iodophenyl)-6-(2-fluorophenyl)-1,2,4,5-tetrazine (12b)

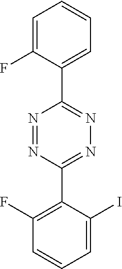 [SMILES: Fc1ccccc1-c1nnc(-c2c(F)cccc2I)nn1]

Rf=0.51 (dichloromethane-heptane=7:3 (v/v)).

¹H NMR (300 MHz, CDCl₃): δ (ppm)=8.46-8.40 (m, 1H), 7.88-7.84 (m, 1H), 7.71-7.63 (m, 1H), 7.46-7.30 (m, 4H).

¹⁹F NMR (282 MHz, CDCl₃): δ (ppm)=−108.8, −111.0.

¹³C NMR (75 MHz, CDCl₃): δ (ppm)=165.1 (bs), 163.9 (d, J=5.9 Hz), 163.6 (d, J=260.7 Hz), 162.3 (d, J=256.6 Hz), 135.7 (d, J=3.7 Hz), 134.8 (d, J=8.8 Hz), 133.8 (d, J=8.8 Hz), 131.9 (d, J=0.8 Hz), 127.3 (d, J=16.4 Hz), 125.1 (d, J=3.9 Hz), 120.5 (d, J=9.8 Hz), 117.8 (d, J=21.6 Hz), 116.6 (d, J=21.5 Hz), 97.4 (d, J=0.9 Hz).

Elemental analysis: Calcd (%) for C₁₄H₇F₂IN₄: C, 42.45, H, 1.78, N, 14.14. Found: C, 42.23, H, 2.29, N, 13.38.

HRMS+p ESI (m/z) [M+H⁺] Calcd for C₁₄H₇F₂IN₄: 396.975. Found: m/z=396.974.

### 3-(2-chloro-6-fluorophenyl)-6-(2-fluorophenyl)-1,2,4,5-tetrazine (12c)

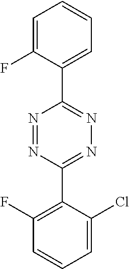 [SMILES: Fc1ccccc1-c1nnc(-c2c(F)cccc2Cl)nn1]

Rf=0.55 (dichloromethane-heptane=7:3 (v/v)).

¹H NMR (300 MHz, CDCl₃): δ (ppm)=8.42 (td, J=7.62, 1.78 Hz, 1H), 7.70-7.63 (m, 1H), 7.59-7.51 (m, 1H), 7.46-7.43 (m, 2H), 7.41-7.23 (m, 2H).

¹⁹F NMR (282 MHz, CDCl₃): δ (ppm)=−111.1, −111.1.

¹³C NMR (75 MHz, CDCl₃): δ (ppm)=163.9 (d, J=5.9 Hz), 163.6 (d, J=260.6 Hz), 163.1 (d, J=255.3 Hz), 162.7 (bs), 135.1 (d, J=3.5 Hz), 134.8 (d, J=8.8 Hz), 133.1 (d, J=9.5 Hz), 131.9 (d, J=0.8 Hz), 126.2 (d, J=3.6 Hz), 125.0 (d, J=3.9 Hz), 122.1 (d, J=17.3 Hz), 120.5 (d, J=9.7 Hz), 117.8 (d, J=21.6 Hz), 115.2 (d, J=21.4 Hz).

Elemental analysis: Calcd (%) for C₁₄H₇ClF₂N₄: C, 55.19, H, 2.32, N, 18.39. Found: C, 55.59, H, 3.20, N, 16.61.

HRMS+p ESI (m/z) [M+H⁺] Calcd for C₁₄H₇ClF₂N₄: 305.040. Found: m/z=305.039.

### 3-(2-bromo-6-chlorophenyl)-6-(2-chlorophenyl)-1,2,4,5-tetrazine (13a)

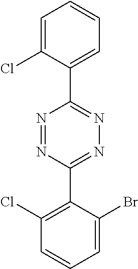 [SMILES: Clc1ccccc1-c1nnc(-c2c(Cl)cccc2Br)nn1]

Rf=0.41 (dichloromethane-heptane=1:1 (v/v)).

¹H NMR (300 MHz, CDCl₃): δ (ppm)=8.15-8.12 (m, 1H), 7.72 (dd, J=8.08, 1.05 Hz, 1H), 7.67-7.52 (m, 4H), 7.43 (t, J=8.11 Hz, 1H).

¹³C NMR (75 MHz, CDCl₃): δ (ppm)=165.9, 165.7, 135.2, 134.1, 134.0, 132.9, 132.7, 132.5, 131.7, 131.6, 131.3, 129.2, 127.5, 124.1.

Elemental analysis: Calcd (%) for C₁₄H₆BrCl₂N₄: C, 44.01, H, 1.85, N, 14.67. Found: C, 44.62, H, 1.98, N, 14.45.

HRMS+p ESI (m/z) [M+Na⁺] Calcd for Cl₄H₆BrCl₂N₄: 402.912. Found: m/z=402.913.

### 3-(2-chloro-6-iodophenyl)-6-(2-chlorophenyl)-1,2,4,5-tetrazine (13b)

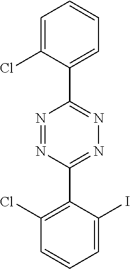 [SMILES: Clc1ccccc1-c1nnc(-c2c(Cl)cccc2I)nn1]

Rf=0.31 (dichloromethane-heptane=1:1 (v/v)).

¹H NMR (300 MHz, CDCl₃): δ (ppm)=8.16-8.13 (m, 1H), 7.76 (dd, J=7.99, 0.97 Hz, 2H), 7.67-7.52 (m, 4H), 7.25 (t, J=8.06 Hz, 1H).

¹³C NMR (75 MHz, CDCl₃): δ (ppm)=167.5, 165.6, 137.9, 137.5, 134.3, 134.1, 133.0, 132.9, 132.5, 131.7, 131.3, 129.9, 127.5, 97.6.

Elemental analysis: Calcd (%) for C₁₄H₇Cl₂IN₄: C, 39.19, H, 1.64, N, 13.06. Found: C, 38.80, H, 1.73, N, 12.78.

HRMS+p ESI (m/z) [M+H⁺] Calcd for C₁₄H₆Cl₂IN₄: 428.916. Found: m/z=428.916.

### 3-(2-bromo-6-fluorophenyl)-6-(2-bromophenyl)-1,2,4,5-tetrazine (14a)

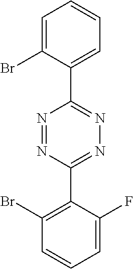 [SMILES: Fc1cccc(Br)c1-c1nnc(-c2ccccc2Br)nn1]

Rf=0.50 (dichloromethane-heptane=1:1 (v/v)).

¹⁹F NMR (282 MHz, CDCl₃): δ (ppm)=−110.0.

### 3-(2,6-dibromophenyl)-6-(2-fluorophenyl)-1,2,4,5-tetrazine (36)

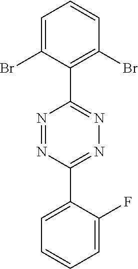 [SMILES: Fc1ccccc1-c1nnc(-c2c(Br)cccc2Br)nn1]

Rf=0.50 (dichloromethane-heptane=1:1 (v/v)).

¹⁹F NMR (282 MHz, CDCl₃): δ (ppm)=−110.9.

### 3-(2-chloro-6-fluorophenyl)-6-(2-chlorophenyl)-1,2,4,5-tetrazine (37)

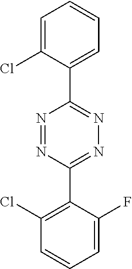 [SMILES: Fc1cccc(Cl)c1-c1nnc(-c2ccccc2Cl)nn1]

Rf=0.39 (dichloromethane-heptane=1:1 (v/v)).

¹H NMR (300 MHz, CDCl₃): δ (ppm)=8.14-8.10 (m, 1H), 7.67-7.64 (m, 1H), 7.62-7.52 (m, 3H), 7.45 (dt, J=8.1, 1.0 Hz, 1H), 7.27 (td, J=8.1, 1.0 Hz, 1H).

¹⁹F NMR (282 MHz, CDCl₃): δ (ppm)=−111.1.

¹³C NMR (75 MHz, CDCl₃): δ (ppm)=165.7, 163.1 (d, J=255.5 Hz), 162.7 (d, J=1.56 Hz), 135.1 (d, J=3.5 Hz), 134.1, 133.2 (d, J=9.6 Hz), 133.0, 132.6, 131.5, 131.4, 127.5, 126.2 (d, J=3.6 Hz), 122.0 (d, J=7.3 Hz), 115.2 (d, J=21.4 Hz).

Elemental analysis: Calcd (%) for C₁₄H₇Cl₂FN₄: C, 52.36, H, 2.20, N, 17.45. Found: C, 53.13, H, 2.74, N, 16.97.

HRMS+p ESI (m/z) [M+H⁺] Calcd for C₁₄H₇Cl₂FN₄: 321.010. Found: m/z=321.010.

### 3-(2,6-dichlorophenyl)-6-(2-fluorophenyl)-1,2,4,5-tetrazine (38)

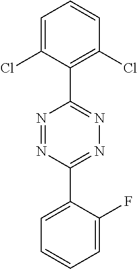 [SMILES: Fc1ccccc1-c1nnc(-c2c(Cl)cccc2Cl)nn1]

Rf=0.39 (dichloromethane-heptane=1:1 (v/v)).

¹⁹F NMR (282 MHz, CDCl₃): δ (ppm)=−111.0.

### 3-(2-bromo-6-fluorophenyl)-6-(2-chloro-6-fluorophenyl)-1,2,4,5-tetrazine (39)

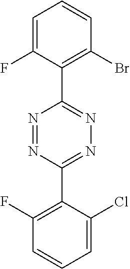 [SMILES: Fc1cccc(Cl)c1-c1nnc(-c2c(F)cccc2Br)nn1]

Rf=0.59 (dichloromethane-heptane=3:2 (v/v)).

¹H NMR (300 MHz, CDCl₃): δ (ppm)=7.64-7.44 (m, 4H), 7.35-7.26 (m, 2H).

¹⁹F NMR (282 MHz, CDCl₃): δ (ppm)=−109.9, −111.0.

¹³C NMR (75 MHz, CDCl₃): δ (ppm)=164.2 (d, J=1.3 Hz), 163.2 (d, J=1.3 Hz), 163.0 (d, J=255.6 Hz), 162.9 (d, J=256.3 Hz), 135.1 (d, J=3.5 Hz), 133.6 (d, J=9.2 Hz), 133.3 (d, J=9.5 Hz), 129.3 (d, J=3.6 Hz), 126.2 (d, J=3.6 Hz), 123.7 (sb), 123.7 (d, J=13.7 Hz), 121.9 (d, J=17.3 Hz), 115.8 (d, J=21.2 Hz), 115.2 (d, J=21.2 Hz).

Elemental analysis: Calcd (%) for C₁₄H₆BrClF₂N₄: C, 43.84, H, 1.58, N, 14.61. Found: C, 44.51, H, 2.02, N, 14.25.

HRMS+p ESI (m/z) [M+H⁺] Calcd for C₁₄H₆BrClF₂N₄: 382.951. Found: m/z=382.951.

### 3-(2-chloro-6-fluorophenyl)-6-(2-fluoro-6-iodophenyl)-1,2,4,5-tetrazine (40)

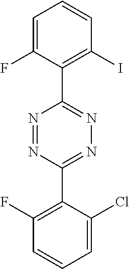 [SMILES: Fc1cccc(Cl)c1-c1nnc(-c2c(F)cccc2I)nn1]

Rf=0.54 (dichloromethane-heptane=1:1 (v/v)).

¹H NMR (300 MHz, CDCl₃): δ (ppm)=7.73 (dd, J=8.08, 1.05 Hz, 1H), 7.64-7.59 (m, 2H), 7.54-7.41 (m, 2H), 7.33 (td, J=8.80, 1.05 Hz, 1H).

¹⁹F NMR (282 MHz, CDCl₃): δ (ppm)=−109.9.

¹³C NMR (75 MHz, CDCl₃): δ (ppm)=166.5, 164.2 (d, J=1.3 Hz), 162.8 (d, J=256.2 Hz), 135.1, 134.0, 133.6 (d, J=9.1 Hz), 132.8, 131.7, 129.2 (d, J=3.7 Hz), 129.1, 123.9, 123.8 (d, J=17.4 Hz), 123.7 (d, J=2.7 Hz), 115.8 (d, J=21.2 Hz).

Elemental analysis: Calcd (%) for C₁₄H₆Br₂ClFN₄: C, 37.83, H, 1.36, N, 12.61. Found: C, 38.57, H, 1.86, N, 11.98.

HRMS+p ESI (m/z) [M+H⁺] Calcd for C₁₄H₆Br₂ClFN₄: 442.870. Found: m/z=442.871.

### 3-(2-bromo-6-chlorophenyl)-6-(2-bromo-6-fluorophenyl)-1,2,4,5-tetrazine (41)

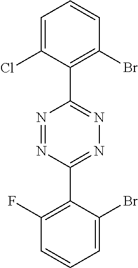 [SMILES: Fc1cccc(Br)c1-c1nnc(-c2c(Cl)cccc2Br)nn1]

Rf=0.54 (dichloromethane-heptane=1:1 (v/v)).

¹H NMR (300 MHz, CDCl₃): δ (ppm)=7.73 (dd, J=8.08, 1.05 Hz, 1H), 7.64-7.59 (m, 2H), 7.54-7.41 (m, 2H), 7.33 (td, J=8.80, 1.05 Hz, 1H).

¹⁹F NMR (282 MHz, CDCl₃): δ (ppm)=−109.9.

¹³C NMR (75 MHz, CDCl₃): δ (ppm)=166.5, 164.2 (d, J=1.3 Hz), 162.8 (d, J=256.2 Hz), 135.1, 134.0, 133.6 (d, J=9.1 Hz), 132.8, 131.7, 129.2 (d, J=3.7 Hz), 129.1, 123.9, 123.8 (d, J=17.4 Hz), 123.7 (d, J=2.7 Hz), 115.8 (d, J=21.2 Hz).

Elemental analysis: Calcd (%) for C₁₄H₆Br₂ClFN₄: C, 37.83, H, 1.36, N, 12.61. Found: C, 38.57, H, 1.86, N, 11.98.

HRMS+p ESI (m/z) [M+H⁺] Calcd for C₁₄H₆Br₂ClFN₄: 442.870. Found: m/z=442.871.

### 3-(2-chloro-6-iodophenyl)-6-(2-fluoro-6-iodophenyl)-1,2,4,5-tetrazine (42)

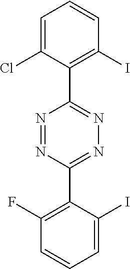 [SMILES: Fc1cccc(I)c1-c1nnc(-c2c(Cl)cccc2I)nn1]

Rf=0.57 (dichloromethane-heptane=1:1 (v/v)).

¹H NMR (300 MHz, CDCl₃): δ (ppm)=7.97 (dd, J=8.0, 1.0 Hz, 1H), 7.89-7.86 (m, 1H), 7.63 (dd, J=8.0, 1.0 Hz, 1H), 7.37-7.33 (m, 2H), 7.27 (t, J=8.10 Hz, 1H).

¹⁹F NMR (282 MHz, CDCl₃): δ (ppm)=−108.62.

¹³C NMR (75 MHz, CDCl₃): δ (ppm)=168.1, 165.6 (d, J=1.4 Hz), 158.8 (d, J=256.9 Hz), 137.9, 137.4, 135.6 (d, J=3.7 Hz), 134.2, 134.0 (d, J=9.7 Hz), 133.1, 129.9, 127.2 (d, J=16.7 Hz), 116.6 (d, J=21.2 Hz), 97.4, 97.3 (d, J=0.9 Hz).

Elemental analysis: Calcd (%) for C₁₄H₆ClFI₂N₄: C, 31.23, H, 1.12, N, 10.40. Found: C, 31.76, H, 1.78, N, 9.63.

HRMS+p ESI (m/z) [M+H⁺] Calcd for C₁₄H₆ClFI₂N₄: 538.843. Found: m/z=538.844.

### 3-(2-chlorophenyl)-6-(2-bromo-6-fluorophenyl)-1,2,4,5-tetrazine (43)

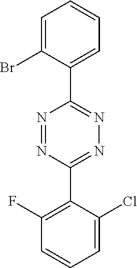 [SMILES: Fc1cccc(Cl)c1-c1nnc(-c2ccccc2Br)nn1]

Rf=0.38 (dichloromethane-heptane=1:1 (v/v)).

¹H NMR (300 MHz, CDCl₃): δ (ppm)=8.08 (dd, J=8.0, 1.0 Hz, 1H), 7.85 (dd, J=8.0, 1.0 Hz, 1H), 7.62-7.43 (m, 4H), 7.27 (td, J=8.10, 1H Hz, 1H).

¹⁹F NMR (282 MHz, CDCl₃): δ (ppm)=−111.1.

¹³C NMR (75 MHz, CDCl₃): δ (ppm)=166.4, 163.0 (d, J=255.5 Hz), 162.6 (d, J=1.4 Hz), 135.1 (d, J=3.5 Hz), 135.0, 133.5, 133.2 (d, J=9.7 Hz), 132.9, 132.5, 128.1, 126.2 (d, J=3.6 Hz), 122.7, 122.0 (d, J=7.3 Hz), 115.2 (d, J=21.3 Hz).

Elemental analysis: Calcd (%) for C₁₄H₇BrClFN₄: C, 46.00, H, 1.93, N, 15.33. Found: C, 46.50, H, 2.05, N, 14.51.

HRMS+p ESI (m/z) [M+H⁺] Calcd for C₁₄H₇BrClFN₄: 364.960. Found: m/z=364.960.

The halogenated and acetylated mono and polyfunctionalized compounds above-described are useful precursors for further organic and organometallic reactions as exemplified below with Suzuki-Miyaura cross-coupling towards ortho-arylated tetrazines 44-45. The exemplification with 44a-e and 45a-b validate a determining interest of the halogenated precursors.

### EXAMPLE 3: FUNCTIONALIZATION OF COMPOUNDS OF FORMULA (I) WITH SUZUKI-MIYAURA CROSS-COUPLING REACTION

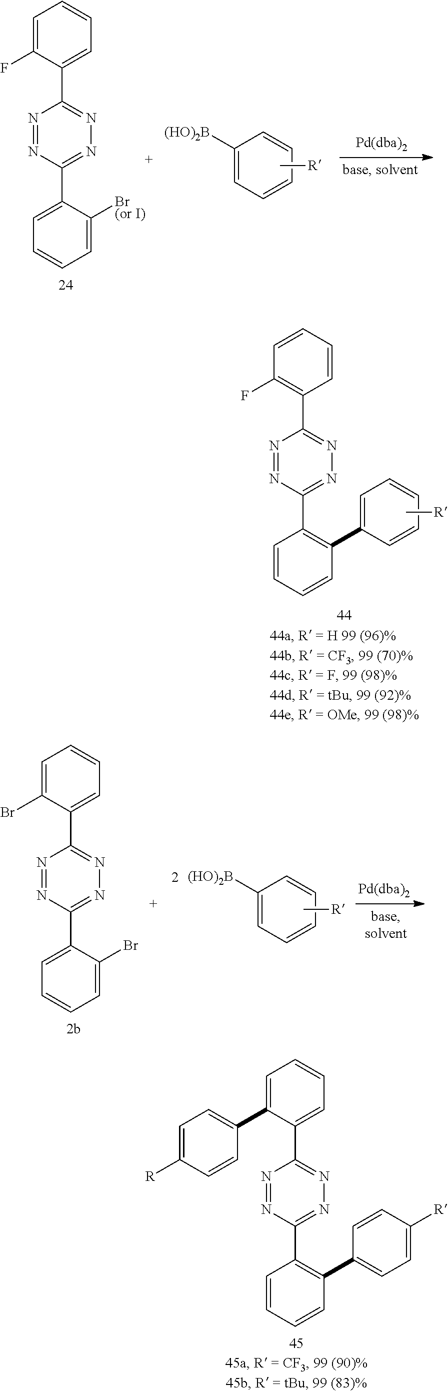

As a typical experiment, 3-(2-bromophenyl)-6-(2-fluorophenyl)-1,2,4,5-tetrazine (23.0 mg, 0.07 mmol), phenylboronic acid (17.1 mg, 0.14 mmol), Pd(dba)₂ (4.0 mg, 0.007 mmol) and K₂CO₃ (19.3 mg, 0.14 mmol) were introduced in a Schlenk tube, equipped with a magnetic stirring bar. Dry toluene (0.7 mL) was added, and the Schlenk tube purged several times with argon. The Schlenk tube was placed in a pre-heated oil bath at 110° C. and reactants were allowed to stir for 5 h. After cooling to room temperature, the reaction mixture was diluted with dichloromethane, and was washed three times with water. The combined organic layer was washed with water and dried over MgSO₄. The solvent was removed in vacuo and the crude product was purified by silica gel column chromatography to afford 20 in 59% (13.6 mg) yield.

### 3-(2-fluorophenyl)-6-[(1,1′-biphenyl)-2-yl]-1,2,4,5-tetrazine (44a)

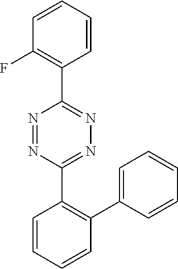 [SMILES: Fc1ccccc1-c1nnc(-c2ccccc2-c2ccccc2)nn1]

Rf=0.50 (dichloromethane-heptane=7:3 (v/v)).

1H NMR (300 MHz, CDCl₃): δ (ppm)=8.24 (td, J=7.65, 1.78 Hz, 1H), 8.13-8.10 (m, 1H), 7.72-7.56 (m, 4H), 7.35 (td, J=7.65, 1.78 Hz, 1H), 7.30-7.26 (m, 4H), 7.16-7.13 (m, 2H).

19F NMR (282 MHz, CDCl₃): δ (ppm)=−112.0.

13C NMR (300 MHz, CDCl₃): δ (ppm)=167.0 (d, J=0.8 Hz), 163.3 (d, J=259.6 Hz), 162.6 (d, J=5.8 Hz), 143.0, 140.5, 134.2 (d, J=8.7 Hz), 131.8, 131.6, 131.4, 131.3, 129.5, 128.6, 128.1, 127.4, 124.9 (d, J=3.9 Hz), 120.8 (d, J=9.9 Hz), 117.6 (d, J=21.6 Hz).

Elemental analysis: Calcd (%) for C20H13FN4: C, 73.16, H, 3.99, N, 17.06. Found: C, 72.64, H, 4.39, N, 16.83.

HRMS+p ESI (m/z) [M+H+] Calcd for C20H13FN₄: 329.120. Found: m/z=329.120.

1. A process for producing a compound of formula (I), 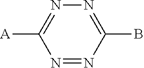
wherein
A is 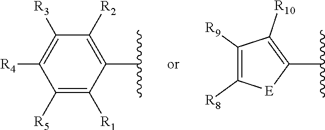
B is 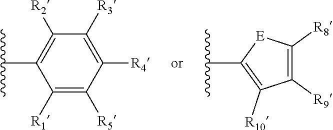
A and B being the same;
R₁, R₁′, R₂ and R₂′ may be the same or different and represent each a hydrogen atom, a halogen atom, a substituted or unsubstituted group selected from alkyl, cycloalkyl, aryl, alkyloxy, cycloalkyloxy, alkyloxycarbonyl, aryloxy, alkylamino, cycloalkylamino, arylamino, provided that at least one of R₁, R₁′, R₂ and R₂′ is a halogen atom or acetate group;
R₃, R₃′, R₄, R₄′, R₅, R₅′ may be the same or different and represent each a hydrogen atom, a halogen atom or a substituted or unsubstituted group selected from alkyl, cycloalkyl, aryl, alkyloxy, cycloalkyloxy, aryloxy, alkylamino, cycloalkylamino, arylamino;
R₈, R₈′, R₉, R₉′ may be the same or different and represent each a hydrogen atom, a halogen atom or a substituted or unsubstituted group selected from alkyl, cycloalkyl, aryl, alkyloxy, cycloalkyloxy, aryloxy, alkylamino, cycloalkylamino, and arylamino;
R₁₀, R₁₀′ may be the same or different and represent each a hydrogen atom or a halogen atom, provided that at least one of R₁₀ and R₁₀′ is a halogen atom or acetate group; and
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
said process comprising:
reacting a compound of formula (II) 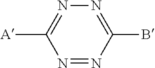
wherein
A′ is 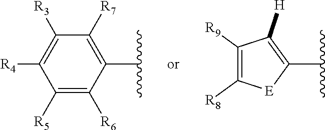
B′ is 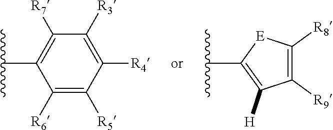
A′ and B′ being the same;
R₆, R₆′, R₇ and R₇′ may be the same or different and represent each a hydrogen atom, a halogen atom, a substituted or unsubstituted group selected from alkyl, cycloalkyl, aryl, alkyloxy, cycloalkyloxy, aryloxy, alkylamino, cycloalkylamino, and arylamino, provided that at least one of R₆, R₆′, R₇ and R₇′ is a hydrogen atom;
R₃, R₃′, R₄, R₄′, R₅, R₅′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino, and arylamino;
R₈, R₈′, R₉, R₉′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino and arylamino; and
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
with an oxidative reagent in presence of a catalyst.

2. The process according to claim 1 for producing a compound of formula (Ia), corresponding to a compound of formula (I)
wherein A is 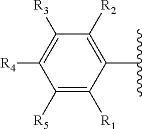
and B is 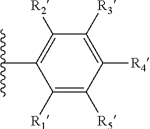
R₁, R₁′, R₂ and R₂′ may be the same or different and represent each a hydrogen atom, a halogen atom, a substituted or unsubstituted group selected from alkyl, cycloalkyl, aryl, alkyloxy, cycloalkyloxy, alkyloxycarbonyle, aryloxy, alkylamino, cycloalkylamino, arylamino, provided that at least one of R₁, R₁′, R₂ and R₂′ is a halogen atom or acetate group; and
R₃, R₃′, R₄, R₄′, R₅, R₅′ may be the same or different and represent each a hydrogen atom, a halogen atom or a substituted or unsubstituted group selected from alkyl, cycloalkyl, aryl, alkyloxy, cycloalkyloxy, aryloxy, alkylamino, cycloalkylamino, arylamino;
said process comprising:
reacting a compound of formula (IIa)
wherein A′ is 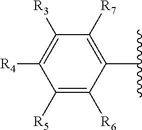
and B′ is 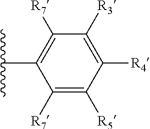
R₆, R₆′, R₇ and R₇′ may be the same or different and represent each a hydrogen atom, a halogen atom, a substituted or unsubstituted group selected from alkyl, cycloalkyl, aryl, alkyloxy, cycloalkyloxy, aryloxy, alkylamino, cycloalkylamino, arylamino, provided that at least one of R₆, R₆′, R₇ and R₇′ is a hydrogen atom; and
R₃, R₃′, R₄, R₄′, R₅ and R₅′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino, arylamino;
with an oxidant in presence of a catalyst.

3. The process according to claim 1 for producing a compound of formula (Ib), corresponding to a compound of formula (I)
wherein A is 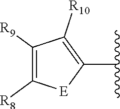
and B is 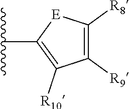
R₈, R₈′, R₉, R₉′ may be the same or different and represent each a hydrogen atom, a halogen atom or a substituted or unsubstituted group selected from alkyl, cycloalkyl, aryl, alkyloxy, cycloalkyloxy, aryloxy, alkylamino, cycloalkylamino, and arylamino;
R₁₀, R₁₀′ may be the same or different and represent each a hydrogen atom or a halogen atom, provided that at least one of R₁₀ and R₁₀′ is a halogen atom or acetate group; and
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
said process comprising:
reacting a compound of formula (IIb)
wherein A′ is 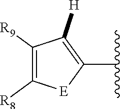
and B′ is 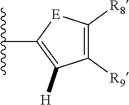
R₈, R₈′, R₉, R₉′ may be the same or different and represent each a hydrogen atom or a substituted or unsubstituted group selected from alkyl, cycloalkyl, aryl, alkyloxy, cycloalkyloxy, aryloxy, alkylamino, cycloalkylamino and arylamino; and
E is an oxygen atom, a sulfur atom or N—R₁₁, wherein R₁₁ is selected from hydrogen, alkyl, cycloalkyl, aryl, alkyloxy, cycloalkyloxy, aryloxy, alkylamino, cycloalkylamino and arylamino;
with an oxidative reagent in presence of a catalyst.

4. The process according to claim 1, wherein the oxidative reagent is selected from the group comprising N-chlorosuccinimide, N-bromosuccinimide, N-iodosuccinimide, N-fluorobenzenesulfonimide and (diacetoxyiodo)benzene.

5. The process according to claim 1, wherein the catalyst is a palladium catalyst.

6. The process according to claim 1, wherein the catalyst is selected from the group comprising palladium(II) catalyst and palladium(0) catalyst.

7. The process according to claim 1, wherein the catalyst is selected from the group comprising palladium acetate, tris(dibenzylideneacetone)dipalladium, bis(dibenzylideneacetone)palladium, allylpalladium(II) chloride dimer and palladium chloride.

8. The process according to claim 1, wherein the process is carried out in presence of a polar solvent.

9. The process according to claim 8, wherein the polar solvent is selected from the group comprising dichloroethane, trifluoromethylbenzene, nitromethane, acetic acid, pivalic acid and propionic acid.

10. The process according to claim 1, wherein the amount of oxidative reagent is ranging from 1 equivalent to 12 equivalent of compound (II).

11. process according to claim 1, wherein the amount of catalyst is ranging from 0.1% to 50%, more preferably 0.1% to 30%, more preferably 0.1% to 20%, more preferably 1% to 50%, more preferably 5% to 20%, more preferably 8% to 15% and more preferably 1% to 15%, in mole to compound (II).

12. The process according to claim 1, wherein the process is performed at a temperature ranging from 80° C. to 150° C., preferably from 90° C. to 130° C., more preferably from 100° C. to 120° C.

13. Compounds of formula (Ia) 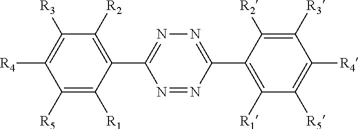
wherein
R₁, R₁′, R₂ and R₂′ may be the same or different and represent each a halogen atom, the halogen atom being the same or different, provided that at least one of R₁, R₁′, R₂ and R₂′ is a different halogen atom compared to the others; and
R₃, R₃′, R₄, R₄′, R₅, R₅′ may be the same or different and represent each a hydrogen atom, an halogen atom or a substituted or unsubstituted group selected from alkyl, cycloalkyl, aryl, alkyloxy, cycloalkyloxy, aryloxy, alkylamino, cycloalkylamino, arylamino.
